(19) United States
(12) Patent Application Publication
(10) Pub. No.: US 2024/0228962 A9
(43) Pub. Date: Jul. 11, 2024

(54) METHODS FOR EXPANDING REGULATORY T CELLS

(71) Applicant: KSQ THERAPEUTICS, INC., Lexington, MA (US)

(72) Inventors: John CHO, Stoneham, MA (US); Kerem Jonatan TUNCEL, Brookline, MA (US)

(21) Appl. No.: 18/547,627

(22) PCT Filed: Feb. 23, 2022
(86) PCT No.: PCT/US2022/017526

§ 371 Date: Aug. 23, 2023

Related U.S. Application Data
(60) Provisional application No. 63/152,787, filed on Feb. 23, 2021.

(51) Int. Cl. C12N 5/0783; A61K 39/00; C12N 9/22; C12N 15/11; C12N 15/90

(52) U.S. Cl. CPC C12N 5/0637; A61K 39/4611; C12N 9/22; C12N 15/11; C12N 15/907; A61K 2239/26; C12N 2501/2302; C12N 2501/51; C12N 2501/515; C12N 2501/53

(57) ABSTRACT

Provided herein are ex vivo methods for expanding regulatory T cells (Tregs), including engineered Tregs, while maintaining their stability and activity. In addition to improving growth and expansion of Tregs, the methods, which can comprise the use of discontinuous stimulation of regulatory T cells (DSORT™), produce Tregs with increased stability and activity.

### CROSS REFERENCE TO RELATED APPLICATIONS

This application claims the priority benefit of U.S. Provisional Application No. 63/152,787, filed Feb. 23, 2021, which is herein incorporated by reference in its entirety.

### REFERENCE TO A SEQUENCE LISTING SUBMITTED ELECTRONICALLY

The Sequence Listing submitted Feb. 23, 2022, as a text file named “4195_022PC01_Seqlisting_ST25,” created on Feb. 22, 2022, and having a size of 11,366 bytes, is hereby incorporated by reference.

### BACKGROUND OF THE DISCLOSURE

### Field

Provided herein are ex vivo methods for expanding regulatory T cells (Tregs), including engineered Tregs, while maintaining their stability and activity.

### Background

Regulatory T cells (Tregs) are critical for preventing autoimmunity. Tregs are CD4⁺ T cells that suppress inflammation and the activity of effector T cells (Teffs) to maintain homeostasis. Defects in the number and/or the function of Tregs are frequently observed in patients with autoimmune disease. Potential therapeutic uses of Tregs have been recognized, and Tregs have been assessed in clinical trials for the treatment of various autoimmune conditions and Graft vs Host Disease (GvHD).

Although clinical studies using freshly isolated Tregs have been conducted in the past, broad use of Treg adoptive cell therapy (ACT) requires ex vivo expansion of Tregs to generate sufficient numbers of cells. The ex vivo expansion needs to be done in such way that the purity, stability, and inherent suppressive functions of Tregs are not compromised.

An inability to effectively expand stable and active populations of Tregs has resulted in significant challenges. Clinical manufacturing of Tregs typically uses enclosed GMP-certified magnetic enrichment systems, such as CliniMACS, for the isolation of CD25 expressing cells, which result in significant contaminations of activated effector T cells (Teffs). The outgrowth of such cells can, at least partially, be prevented by adding rapamycin, an mTOR protein kinase inhibitor, to the cultures. While Tregs are relatively resistant to mTOR inhibition, studies have found that rapamycin reduces the expansion of Tregs over a 35-day period by ˜35%. Moreover, rapamycin influences the phenotype of Tregs, e.g., by altering the expression of tissue-homing receptors, such as CCR4 and CXCR3, as well as their function (e.g., CTLA-4, LAP) and lineage (Helios and Foxp3), which may obscure or alter the effect of gene editing.

Rapamycin is not the only compound that has been used to improve purity or induce growth of Tregs in vitro. TGF-β together with all-trans retinoic acid (ATRA), a vitamin A derivate, have been shown to support growth of Tregs ex vivo in pre-clinical studies, albeit with significant impacts to Treg phenotype, such as upregulation of gut homing receptors like CCR9 and integrin-α4β7, as well as CD161.

An alternative approach for using rapamycin and ATRA is to start from a more

homogenous population of naive CD45RA⁺ naive Tregs harvested from thymus, peripheral blood, or umbilical cord blood. Such isolated products contain fewer effector T cells and therefore are more suitable for long-term Treg expansions in the absence of rapamycin. Ex vivo expanded naive Tregs have been shown to maintain a naïve phenotype and to express lymph node (LN) homing markers such as CD62L and CCR7, which have been shown to be necessary for the prevention of autoimmune disease in preclinical models. However, time is one of the most important factors when it comes to Treg purity and stability. Tregs become suppressive when activated in vitro and maintain a high level of suppressive capacity throughout the first 1-2 weeks in culture, during which time Tregs effectively prevent the outgrowth of contaminating T effector cells. However, over time, the ability of Tregs to control the growth of other T cells is diminished, resulting in rapid accumulation of non-Tregs and the loss of a pure Treg population.

Thus, currently available protocols for ex vivo expansion of Tregs result in poor growth and/or stability. Therefore, a need exits for efficient methods of expanding Tregs to produce pure, stable and highly functional populations of Tregs.

### BRIEF SUMMARY OF THE INVENTION

Methods of expanding regulatory T cells (Tregs) that maximize the growth of Tregs in the first 1-2 weeks of culture and take advantage of the enhanced suppressive activity of Tregs early after activation are provided herein. As demonstrated herein, discontinuous stimulation of regulatory T cells (DSORT™) can achieve rapid Treg expansion by using alternating periods of stimulation and resting. An exemplary DSORT™ process is depicted in FIG. 8. Despite expansion rates over 1000-fold, such methods can produce a pure, stable, and highly functional Treg product. Advantageously, these methods do not require the use of rapamycin to slow down the growth of cells during the first 1-2 weeks of culture.

Provided herein is a method for expanding a population of regulatory T cells (Tregs), the method comprising: (a) a first stimulating step comprising culturing a population of Tregs in the presence of a first stimulatory agent to produce a first stimulated population of Tregs; and (b) a first resting step comprising continuing to culture the first stimulated population of Tregs in the absence of a stimulatory agent to produce a first rested population of Tregs. In some aspects, the method further comprises (c) a second stimulating step comprising culturing the first rested population of Tregs in the presence of a second stimulatory agent to produce a second stimulated population of Tregs. In some aspects, the method further comprises (d) a second resting step comprising continuing to culture the second stimulated population of Tregs in the absence of a stimulatory agent to produce a second rested population of Tregs. In some aspects, the method further comprises (e) a third stimulating step comprising culturing the second rested population of Tregs in the presence of a third stimulatory agent to produce a third stimulated population of Tregs. In some aspects, the method further comprises (f) a third resting step comprising continuing to culture the third stimulated population of Tregs in the absence of a stimulatory agent to produce a third rested population of Tregs. In some aspects, the method further comprises one or more additional stimulating step(s) to produce a further stimulated population of Tregs and/or one or more additional resting step(s) to produce a further rested population of Tregs.

In some aspects, the method further comprises genetically engineering the first rested population of Tregs, the second stimulated population of Tregs, the second rested population of Tregs, the third stimulated population of Tregs, the third rested population of Tregs, the further stimulated population of Tregs, or the further rested population of Tregs.

In some aspects, the method further comprises harvesting the first rested population of Tregs, the second stimulated population of Tregs, the second rested population of Tregs, the third stimulated population of Tregs, the third rested population of Tregs, the further stimulated population of Tregs, the further rested population of Tregs, or the genetically engineered population of Tregs.

Provided herein is a method for expanding a population of regulatory T cells (Tregs), the method comprising: (a) a stimulating step comprising culturing a population of Tregs in a media comprising a stimulatory agent to produce a first stimulated population of Tregs; and (b) washing the stimulated population of Tregs to remove the media comprising the stimulatory agent to produce a washed population of Tregs; and (c) culturing the washed population of Tregs in fresh media to produce a first rested population of Tregs. In some aspects, the fresh media does not comprise any stimulatory agent. In some aspects, the method further comprises genetically engineering the first rested population of Tregs. In some aspects, the method further comprises culturing the first ressted population of Tregs in a media comprising a stimulatory agent to produce a second stimulated population of Tregs.

Provided herein is a method for expanding a population of regulatory T cells (Tregs), the method comprising: (a) a resting step comprising culturing a previously stimulated population of Tregs in the absence of a stimulatory agent to produce a first rested population of Tregs; and (b) a stimulating step comprising adding a stimulatory agent to the first rested population of Tregs to produce a stimulated population of Tregs.

In some aspects, the method further comprises genetically engineering the Tregs. In some aspects, the method further comprises genetically engineering the first rested population of Tregs.

In some aspects, the method further comprises culturing the engineered population of Tregs.

In some aspects, the culturing of the engineered population of Tregs occurs in the presence of a stimulatory agent. In some aspects, the method further comprises continuing to culture the engineered population of Tregs in the absence of the stimulatory agent.

In some aspects, the culturing of the engineered population of Tregs occurs in the absence of the stimulatory agent. In some aspects, the method further comprises continuing to culture the engineered population of Tregs in the presence of a stimulatory agent.

Provided herein is a method for expanding a population of regulatory T cells (Tregs), the method comprising: (a) genetically engineering a population of Tregs to produce an engineered population of Tregs; (b) a stimulating step comprising culturing the engineered population of Tregs in the presence of a stimulatory agent to produce a stimulated engineered population of Tregs; and (c) a resting step comprising continuing to culture the stimulated engineered population of Tregs in the absence of a stimulatory agent to produce a rested engineered population of Tregs.

Provided herein is a method for expanding a population of regulatory T cells (Tregs), the method comprising: (a) genetically engineering a population of Tregs to produce a engineered population of Tregs; (b) a resting step comprising culturing the engineered population of Tregs in the absence of a stimulatory agent to produce a rested engineered population of Tregs; and (c) a stimulating step comprising culturing the rested engineered population of Tregs in the presence of a stimulatory agent to produce a stimulated engineered population of Tregs.

In some aspects, prior to being genetically engineered, the population of Tregs is subject to a stimulating step comprising culturing the population in the presence of a stimulating agent.

In some aspects, prior to being genetically engineered, the population of Tregs is subject to a stimulating step comprising culturing the population in the presence of a simulating agent and then a resting step comprising culturing the population in the absence of a stimulating agent.

In some aspects, prior to being genetically engineered, the population of Tregs is subject to a stimulating step comprising culturing the population in the presence of a simulating agent, then a resting step comprising culturing the population in the absence of a stimulating agent, and then another stimulating step comprising culturing the population in the presence of a stimulating agent.

In some aspects, the genetic engineering occurs in the absence of a stimulatory agent.

In some aspects, the method further comprises harvesting the population of Tregs.

In some aspects, the method further comprises obtaining the population of Tregs from a subject.

In some aspects, the method further comprises obtaining the population of Tregs from thymus, peripheral blood, umbilical cord blood, or a tissue sample of a subject.

In some aspects, the method further comprises obtaining the population of Tregs from peripheral blood from a subject prior to the culturing of step (a).

In some aspects, the population of Tregs was obtained from thymus, peripheral blood, umbilical cord blood, or a tissue sample from a subject. In some aspects, the population of Tregs was obtained from peripheral blood from the subject.

In some aspects, the subject is human.

In some aspects, a tetrameric antibody complex is the stimulating agent in at least one of the stimulating steps and/or the culturing of the engineered population. In some aspects, the tetrameric antibody complex specifically binds to CD3, CD28, CD2, or a combination thereof.

In some aspects, an anti-CD3 antibody or antigen-binding fragment thereof and/or an anti-CD28 antibody or antigen-binding fragment thereof is the stimulating agent in at least one of the stimulating steps and/or the culturing of the engineered population.

In some aspects, CD3-binding and/or CD28-binding supermagnetic beads are the stimulating agent in at least one of the stimulating steps and/or the culturing of the engineered population.

In some aspects, the method does not use supermagnetic beads.

In some aspects, the same stimulatory agent is used throughout the method. In some aspects, at least two different stimulatory agents are used in the method.

In some aspects, the stimulatory agent is present at the same concentration throughout all of the stimulating steps of the method. In some aspects, at least two different concentrations of stimulatory agent are used in the method.

In some aspects, the Tregs are cultured in the presence of IL-2. In some aspects, IL-2 concentration is reduced during the method. In some aspects, IL-2 is present at a concentration of about 800 units/mL for about 7 days and then at about 300 units/mL.

In some aspects, the population of Tregs is cultured in the presence of a stimulatory agent for about 1 to about 5 days, then cultured in the absence of a stimulatory agent for about 1 to about 5 days, then cultured in the presence of a stimulatory agent for about 1 to about 5 days, and then genetically engineered.

In some aspects, the population of Tregs is cultured in the presence of a stimulatory agent for about 3 to about 4 days, then cultured in the absence of a stimulatory agent for about 3 to about 4 days, then cultured in the presence of a stimulatory agent for about 1 to about 4 days, and then genetically engineered. In some aspects, the population of Tregs is cultured in the presence and/or absence of a stimulating agent according to Table A.

In some aspects, the Tregs are cultured in the presence of N-Acetyl-L-cysteine. In some aspects, the N-Acetyl-L-cysteine is present at a concentration of about 5 mM in the culture.

In some aspects, the population of Tregs is genetically engineered when the population of Tregs has expanded about 250-fold. In some aspects, the population of Tregs is genetically engineered about 6 to about 10 days after Tregs were obtained from a subject. In some aspects, the population of Tregs is genetically engineered about 7 days after Tregs were obtained from a subject.

In some aspects, the genetic engineering comprises introducing a nucleic acid into the population of Tregs. In some aspects, the nucleic acid is a viral nucleic acid. In some aspects, the nucleic acid is not a viral nucleic acid. In some aspects, the nucleic acid encodes a protein. In some aspects, the protein is a heterologous protein. In some aspects, the heterologous protein is a chimeric antigen receptor (CAR).

In some aspects, the genetic engineering comprises introducing a gene-regulating system into the population of Tregs. In some aspects, the gene-regulating system comprises (i) a nucleic acid molecule; (ii) an enzymatic protein; or (iii) a nucleic acid molecule and an enzymatic protein. In some aspects, the gene-regulating system comprises a nucleic acid molecule selected from an siRNA, an shRNA, a microRNA (miR), an antagomiR, or an antisense RNA. In some aspects, the gene-regulating system comprises an enzymatic protein, and wherein the enzymatic protein has been engineered to specifically bind to a target sequence in one or more genes in the Tregs. In some aspects, the enzymatic protein is a Transcription activator-like effector nuclease (TALEN), a zinc-finger nuclease, or a meganuclease. In some aspects, the gene-regulating system comprises a nucleic acid molecule and an enzymatic protein, wherein the nucleic acid molecule is a guide RNA (gRNA) molecule and the enzymatic protein is a Cas protein or Cas ortholog. In some aspects, the Cas protein is a Cas9 protein.

In some aspects, the introducing uses electroporation or Ribonucleoprotein (RNP)-mediated methods.

In some aspects, the method does not use an artificial antigen presenting cell.

In some aspects, the method does not use rapamycin. In some aspects, the method comprises using rapamycin.

In some aspects, the method increases the number of Tregs by at least 1000-fold in 11 days.

In some aspects, the method results in Tregs with a smaller surface area than Tregs that are cultured in the presence of a stimulating agent for 6 days. In some aspects, the method results in an increased proportion of Helios+Foxp3+ Tregs as compared to Tregs that are cultured in the presence of a stimulating agent for 6 days. In some aspects, the method results in Tregs with an increased ability to suppress proliferation of effector T cells (Teffs) as compared to Tregs that are cultured in the presence of a stimulating agent for 6 days. In some aspects, the increased ability to suppress proliferation of Teffs is at least an 8-fold increased ability.

In some aspects, the method prevents overstimulation of the population of Tregs.

In some aspects, the method reduces activation-induced cell death as compared to Tregs that are cultured in the presence of a stimulating agent for 6 days.

In some aspects, at least 75% of Helios expression in the Tregs is maintained.

In some aspects, the Tregs are Helios+.

In some aspects, the Tregs have a fully demethylated Treg-specific demethylated region (TSDR).

In some aspects, the method further comprises cryopreserving the population of Tregs.

Provided herein is a population of Tregs produced by any provided herein. In some aspects, provided herein is a cryoprsereved population of Tregs produced by any method provided herein.

In some aspects, the method further comprises administering the population of Tregs to a subject.

Provided herein is a method of treating an autoimmune or inflammatory disease in a subject comprising administering to the subject an effective amount a population of Tregs obtained using any method provided herein or any Tregs provided herein.

Provided herein is a method of treating or preventing graft vs host disease (GVHD) in a subject comprising administering to the subject an effective amount a population of Tregs obtained using any method provided herein or any Tregs provided herein.

Provided herein is a method of decreasing an immune response in a subject comprising administering to the subject an effective amount a population of Tregs obtained using any method provided herein or any Tregs provided herein.

In some aspects, the population of Tregs is allogeneic to the subject. In some aspects, the population of Tregs is autologous to the subject.

### BRIEF DESCRIPTION OF THE DRAWINGS/FIGURES

FIG. 1 shows the expansion of regulatory T cells (Tregs) subjected to continuous or discontinuous stimulation with either CD3/28/2 tetrameric monoclonal antibodies (mAbs) or Dynabeads. (See Example 2.)

FIG. 2 shows relative size, as a measure of activation, of Tregs subjected to continuous (“standard”) or discontinuous (“DSORT™”) stimulation. (See Example 3.)

FIG. 3 shows the expansion of Tregs subjected to continuous stimulation or discontinuous stimulation with an extended non-stimulatory phase. Stimulations were performed with either CD3/28/2 tetrameric monoclonal antibodies (mAbs) or Dynabeads. (See Example 4.)

FIG. 4 shows the expansion of CRISPR-engineered Tregs subjected to continuous (“Cont”) stimulation or discontinuous (DSORT™) stimulation. Open squares represent mean values of n=3. (See Example 5.)

FIG. 5 provides a graph (top) showing the number of cells (left y-axis) and fold-expansion (right y-axis) of Tregs obtained using DSORT™ and a table (bottom) showing the fold-expansion obtained using DSORT™ (“KSQ Tx”) in addition to alternative expansion protocols. “Thiel” study refers to THEIL, A., et al., “Adoptive transfer of allogeneic regulatory T cells into patients with chronic graft-versus-host disease,” Cytotherapy 17(4):P473-486 (April 2015). “Bluestone” study refers to BLUESTONE, J. A., et al., “Type 1 diabetes immunotherapy using polyclonal regulatory T cells,” Sci Transl med 7(315):315ra189 (November 2015). “Canavan” study refers to CANAVAN, J. B., et al., “Developing in vitro expanded CD45RA+ regulatory T cells as an adoptive cell therapy for Crohn's disease,” Gut 65(4):584-594 (published online February 2015). “Safina” study refers to SAFINIA, N., et al., “Successful expansion of functional and stable regulatory T cells for immunotherapy in liver transplantation,” Oncotarget 7(7):7563-7577 (February 2016). “Trzonkowska” study refers to MAREK-TRZONKOWSKA, N., et al., “Mild hypothermia provides Treg stability,” Scientific Reports 7:11915 (September 2017). “Lombardi” study refers to FRASER, H., et al., “A Rapamycin-Based GMP-Compatible Process for the Isolation and Expansion of Regulatory T Cells for Clinical Trials,” Molecular Therapy—Methods & Clinical Development 8:198-209 (January 2018). “Leventhal” study refers to MATHEW, J. M., et al., “A Phase I Clinical Trial with Ex Vivo Expanded Recipient Regulatory T cells in Living Donor Kidney Transplants,” Scientific Reports 8:7428 (May 2018). “Thonhoff” study refers to THONHOFF, J. R., et al., “Expanded autologous regulatory T-lymphocyte infusions in ALS,” Neurology Neuroimmunology & Neuroinflammation 5(4):1-7 (July 2018). “MacDonald” study refers to MACDONALD, K. N., et al., “Cryopreservation timing is a critical process parameter in a thymic regulatory T-cell therapy manufacturing protocol,” Cytotherapy 21(12):1216-1233 (December 2019). (See Example 6.)

FIG. 6 (A-C) shows (a) the methylation status of a Treg-specific demethylated region (TSDR) in cells with and without Helios and Foxp3 expression, (b) the ability of cell populations with varying proportions of cells expressing Helios to suppress proliferation of effector T cells, and (c) Helios expression is cells expanded using discontinuous (“DSORT™”) or continuous (“standard”) stimulation. (See Example 7.)

FIG. 7 shows the ability of Tregs subjected to continuous stimulation (standard expansion) or discontinuous stimulation (DSORT™) to suppress proliferation of CD4⁺ effector T cells. (See Example 8.)

FIG. 8 is a schematic of an exemplary DSORT™ process in which Tregs are subjected to alternating stimulating and resting cycles and genetically engineered. “Flask” indicates that Tregs are moved from a plate to a flask.

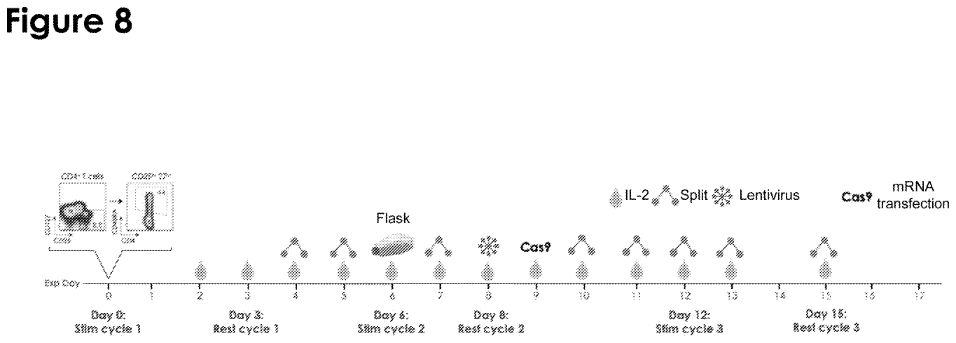

### DETAILED DESCRIPTION OF THE INVENTION

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

It is understood that wherever aspects are described herein with the language “comprising,” otherwise analogous aspects described in terms of “consisting of” and/or “consisting essentially of” are also provided.

As used herein, the singular form “a,” “an,” and “the” includes plural references unless indicated otherwise.

Use of the term “or” herein is not meant to imply that alternatives are mutually exclusive. In this application, the use of “or” means “and/or” unless expressly stated or understood by one skilled in the art. In the context of a multiple dependent claim, the use of “or” refers back to more than one preceding independent or dependent claim. The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The term “about,” as used herein, includes the recited number ±10%. Thus, “about 10” means 9 to 11. As is understood by one skilled in the art, reference to “about” a value or parameter herein includes (and describes) instances that are directed to that value or parameter per se. For example, description referring to “about X” includes description of “X.”

As used herein, the term “regulatory T cells” or “Tregs” refers to a subpopulation of T cells, constitutively expressing the forkhead box P3 (Foxp3) transcription factor, which modulates the immune system, maintains tolerance to self-antigens, and abrogates autoimmune and inflammatory diseases. These cells generally suppress or downregulate induction and proliferation of effector T cells and modulate antigen presenting cell function. Tregs are cells capable of suppressive activity (i.e. inhibiting proliferation of conventional T cells), either by cell-cell contact or through the release of immunosuppressive cytokines.

As used herein, the term “population” refers to a population of cells, wherein the majority, i.e., at least 50% (optionally at least 60%, at least 70%, or at least about 80%) of the total number of cells have the specified characteristics (e.g., functional characteristics and/or markers of interest) of the cells of interest. Thus, a “population of Tregs” refers to a population of cells, wherein the majority of the cells are Tregs, but some cells can be cells that are not Tregs (e.g., some cells can be Teffs).

As used herein, “culturing” refers to growing one or more cells in vitro under defined or controlled conditions. Examples of culturing conditions which can be defined include temperature, gas mixture, time, and medium formulation.

As used herein, the terms “culture medium” and “cell culture medium” and “feed medium” and “fermentation medium” refer to a nutrient solutions used for growing and or maintaining cells, especially mammalian cells. Without limitation, these solutions ordinarily provide at least one component from one or more of the following categories: (1) an energy source, usually in the form of a carbohydrate such as glucose; (2) all essential amino acids, and usually the basic set of twenty amino acids; (3) vitamins and/or other organic compounds required at low concentrations; (4) free fatty acids or lipids, for example linoleic acid; and (5) trace elements, where trace elements are defined as inorganic compounds or naturally occurring elements that are typically required at very low concentrations, usually in the micromolar range. The nutrient solution can be supplemented electively with one or more components from any of the following categories: (1) hormones and other growth factors such as, serum, insulin, transferrin, and epidermal growth factor; (2) salts, for example, magnesium, calcium, and phosphate; (3) buffers, such as HEPES; (4) nucleosides and bases such as, adenosine, thymidine, and hypoxanthine; (5) protein and tissue hydrolysates, for example peptone or peptone mixtures which can be obtained from purified gelatin, plant material, or animal byproducts; (6) antibiotics, such as gentamycin; (7) cell protective agents, for example pluronic polyol; and (8) galactose. Commercially available media such as Ham's F10 (Sigma), Minimal Essential Medium ((MEM), (Sigma)), RPMI-1640 (Sigma), and Dulbecco's Modified Eagle's Medium ((DMEM), (Sigma)) are suitable for culturing the host cells. In addition, any of the media described in Ham et al., Meth. Enz. 58:44 (1979), Barnes et al., Anal. Biochem.102:255 (1980) can be used as culture media for the host cells. Any other necessary supplements can also be included at appropriate concentrations.

As used herein a “stimulatory agent” or a “stimulating agent” refers to an agent that activates (directly or indirectly) a T cell receptor and/or a costimulatory molecule, such as CD28 or GITR, on a Treg and/or that has mitogenic activity. Stimulatory agents can increase proliferation of Tregs. The ability of an agent to act as a stimulatory agent can be demonstrated by its ability to increase phosphorylation of ITAM motifs on the CD3 zeta subunit.

As used herein, culturing in the “presence of a stimulatory agent” or the “presence of a stimulating agent” refers to culturing Tregs in the presence of a sufficient amount of stimulatory agent to activate T cell receptors and/or co-stimulatory molecules, such as CD28 or GITR, on the Tregs.

As used herein, culturing in the “absence of a stimulatory agent” or the “absence of a stimulating agent” can refer to culturing in the absence of a sufficient amount of stimulatory agent to activate T cell receptors and/or co-stimulatory molecules, such as CD28 or GITR, on the Tregs. Culturing in the “absence of a stimulatory agent” includes culturing without any stimulatory agent.

The term “inoculation” as used herein refers to the addition of cells to culture medium to start the culture.

As used herein the term “genetically engineering” refers to a process of altering (the addition, suppression or substitution) the genetic makeup (i.e., of at least one nucleic acid) of a cell by using biotechnology to directly manipulate a nucleic acids in the cell. Genetically engineering includes, for example, introducing heterologous nucleic acids into a cell or population of cells. Nucleic acids can be introduced into a cell or population of cells, e.g., by transduction or transfection.

The term “transduction” or “transducing” refers to the viral transfer of genetic material and its expression in a recipient cell.

The term “transfection” or “transfecting” as used herein refers to the process of introducing DNA (e.g., formulated DNA expression vector) into a cell, thereby, allowing cellular transformation.

As used herein, the term “vector” refers to a nucleic acid molecule allowing insertion of foreign nucleic acid without disrupting the ability of the vector to replicate and/or integrate in a host cell.

The term “recombinant vector” as used herein refers to a polynucleotide molecule capable transferring or transporting another polynucleotide inserted into the vector. The inserted polynucleotide may be an expression cassette. In some aspects, a recombinant vector may be viral vector or a non-viral vector (e.g., a plasmid).

An “expression cassette” or “expression construct” refers to a DNA polynucleotide sequence operably linked to a promoter. “Operably linked” refers to a juxtaposition wherein the components so described are in a relationship permitting them to function in their intended manner. For instance, a promoter is operably linked to a polynucleotide sequence if the promoter affects the transcription or expression of the polynucleotide sequence.

As used herein, the terms “expression” and “expresses” are used to refer to transcription and translation occurring within a cell. The level of expression of a product gene in a host cell can be determined on the basis of either the amount of corresponding mRNA that is present in the cell or the amount of the protein encoded by the product gene that is produced by the cell, or both.

The term “sample” refers to a biological composition (e.g., a cell or a portion of a tissue). In some aspects, a sample is a “primary sample” in that it is obtained directly from a subject; in some aspects, a “sample” is the result of processing of a primary sample, for example to remove certain components and/or to isolate or purify certain components of interest. In some aspects, the sample is blood sample.

The terms “polypeptide” and “protein” are used interchangeably to refer to a polymer of amino acid residues and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain the antigenic determining regions of an intact antibody (e.g., the complementarity determining regions (CDR)). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some aspects, the variable region is a human variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs).

The terms “VL” and “VL domain” are used interchangeably to refer to the light chain variable region of an antibody. The terms “VH” and “VH domain” are used interchangeably to refer to the heavy chain variable region of an antibody.

In some aspects, an antigen-binding fragment of an antibody is an scFv. As used herein, the term “single-chain variable fragment” or “scFv” is a fusion protein of the variable regions of the heavy (VH) and light chains (VL) of an immunoglobulin. The heavy (VH) and light chains (VL) are either joined directly or joined by a peptide-encoding linker (e.g., 10, 15, 20, or 25 amino acids), which connects the N-terminus of the VH with the C-terminus of the VL, or the C-terminus of the VH with the N-terminus of the VL. The linker can be rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including VH- and VL-encoding sequences.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the aspects being described, single-stranded (such as sense or antisense) and double-stranded polynucleotides.

The term “engineered antigen receptor” refers to a non-naturally occurring antigen-specific receptor such as a chimeric antigen receptor (CAR) or a recombinant T cell receptor (TCR). The term “Chimeric Antigen Receptor” or “CAR” has its general meaning in the art and refers to an artificially constructed hybrid polypeptide containing an antigen binding domain, e.g., of an antibody (e.g., scFv), linked to a T-cell signaling domain. The antigen binding domain and the T cell signaling domain can be linked via a hinge. “TCR” has its general meaning in the art and refers to protein complexes that recognize a particular target and that comprise TCRα and/or TCRβ chains.

The term “hinge” or “ hinge region ” refers to a flexible connector region, e.g. natural or synthetic polypeptides, providing structural flexibility and spacing to flanking polypeptide regions.

The term “modified” refers to a substance or compound (e.g., a cell, a polynucleotide sequence, and/or a polypeptide sequence) that has been altered or changed as compared to the corresponding unmodified substance or compound.

The term “naturally-occurring” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is naturally occurring.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects, an antibody, polynucleotide, vector, cell, or composition which is isolated is substantially pure. As used herein, “substantially pure” refers to material which is at least 50% pure (i.e., free from contaminants). “Substantially pure” therefore also includes materials that are at least 90% pure, at least 95% pure, at least 98% pure, or at least 99% pure.

The term “in vivo” refers to an event that takes place in a mammalian subject's body and does not refer to events that take place in cell cultures (including mammalian cell cultures).

The term “ex vivo” refers to an event that takes place outside of a mammalian subject's body, in an artificial environment.

The term “in vitro” refers to an event that takes places in a test system. In vitro assays encompass cell-based assays in which alive or dead cells may be employed and may also encompass a cell-free assay in which no intact cells are employed.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

As used herein, “delaying” means to defer, hinder, slow, retard, stabilize, suppress and/or postpone development or progression of the disease. This delay can be of varying lengths of time, depending on the history of the disease and/or individual being treated.

A “therapeutically effective amount” of a substance can vary according to factors such as the disease state, age, sex, and weight of the individual, and the ability of the substance to elicit a desired response in the individual. A therapeutically effective amount is also one in which any toxic or detrimental effects of the substance are outweighed by the therapeutically beneficial effects. A therapeutically effective amount can be delivered in one or more administrations. A therapeutically effective amount refers to an amount effective, at dosages and for periods of time necessary, to achieve the desired therapeutic effect.

A “prophylactically effective amount” refers to an amount effective, at dosages and for periods of time necessary, to achieve a desired prophylactic result (e.g., prevention of infection or disease onset).

“Decrease” or “reduce” refers to a decrease or a reduction in a particular value of at least 5%, for example, a 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100% decrease as compared to a reference value.

“Increase” refers to an increase in a particular value of at least 5%, for example, a 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, 100%, 200%, 300%, 400%, 500%, or more increase as compared to a reference value.

The terms “administer,” “administering,” “administration,” and the like refer to methods that can be used to enable delivery of the therapeutic agent to the desired site of biological action. Administration techniques that can be employed with the agents and methods described herein are found in e.g., Goodman and Gilman, The Pharmacological Basis of Theraputics, current ed.; Pergamon; and Remington's, Pharmaceutical Sciences (current edition), Mack Publishing Co., Easton, Pa. Administration of two or more therapeutic agents includes simultaneous (concurrent) and consecutive administration in any order.

The terms “individual” or “subject” are used interchangeably herein to refer to an animal, for example, a mammal, such as a human. In some instances, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some instances, the subject to receive the treatment can be a patient, designating the fact that the subject has been identified as having a disorder of relevance to the treatment, or being at particular risk of contracting the disorder.

For the purposes of the present disclosure, regulatory T cells used in therapy can be isolated from the subject they later administered to (“autologous cells”) or from another individual (“allogeneic cells”). As used herein, “allogeneic cells” refers to cells isolated from one subject (the donor) and infused in another (the recipient or host). As used herein, “autologous cells” refers to cells that are isolated and infused back into the same subject (recipient or host).

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998).

### Tregs and Population of Tregs

Tregs expressing the transcription factor forkhead box P3 (FOXP3) are naturally present in the immune system. Accordingly, in some aspects provided herein a Treg is a FoxP3⁺CD4⁺ T cell. The transcription factor Helios is also expressed in many Tregs naturally present in the immune system. Accordingly, in some aspects provided herein, a Treg is a FoxP3⁺Helios⁺ or a FoxP3⁺CD4⁺Helios⁺ T cell.

In some aspects, a Treg has a fully demethylated Treg-specific demethylated region (TSDR).

FoxP3, Helios and the demethylation of TSDR are intracellular Treg markers. However, cell surface markers including CD25^{hi} (high molecular density) and CD127^{lo} (low molecular density) can also serve as markers of Tregs and can be used to identify Tregs, e.g., using flow cytometry. Accordingly, in some aspects a Treg is a CD25⁺ T cell. In some aspects, a Treg is a CD25⁺CD4⁺ T cell. In some aspects, a Treg is CD25^{hi}CD4⁺ T cell. In some aspects a Treg is a CD127^{lo} T cell. In some aspects, a Treg is CD127^{lo}CD4⁺ T cell. In some aspects, a Treg is CD25^{hi}CD127^{lo}CD4⁺ T cell.

In some aspects a Treg is a CD25^{lo}FoxP3⁺ T cell. In some aspects, a Treg is a CD25⁺CD4⁺FoxP3⁺ T cell. In some aspects, a Treg is CD25^{hi}CD4⁺FoxP3⁺ T cell. In some aspects a Treg is a CD127^{lo}FoxP3⁺ T cell. In some aspects, a Treg is CD127^{lo}CD4⁺FoxP3⁺ T cell. In some aspects, a Treg is CD25^{hi}CD127^{lo}CD4⁺FoxP3⁺ T cell.

In some aspects, a Treg is a CD45RA^{hu}CD25^{hi}CD127^{lo}CD4⁺ T cell. In some aspects, a Treg is a CD45RA^{hi}CD25^{hi}CD127^{lo}CD4⁺FoxP3⁺ T cell.

In some aspects a Treg is a CD25⁺FoxP3⁺Helios⁺ T cell. In some aspects, a Treg

is a CD25⁺CD4⁺FoxP3⁺Helios⁺ T cell. In some aspects, a Treg is CD25^{hi}CD4⁺FoxP3⁺Helios⁺ T cell. In some aspects a Treg is a CD127^{lo}FoxP3⁺Helios⁺ T cell. In some aspects, a Treg is CD127^{lo}CD4⁺FoxP3⁺Helios⁺ T cell. In some aspects, a Treg is CD25^{hi}CD127^{lo}CD4⁺FoxP3⁺Helios⁺ T cell.

In some aspects, a Treg is a CD45RA^{hi}CD25^{hi}CD127^{lo}CD4⁺Helios⁺ T cell. In some aspects, a Treg is a CD45RA^{hi}CD25^{hi}CD127^{lo}CD4⁺FoxP3⁺Helios⁺ T cell.

In some aspects, a Treg expresses SOCS, PD-1, CLTA4, neuropilin, TRAIL, and/or GITR.

In some aspects, a Treg expresses IL-10 and/or TGFP.

In some aspects, the Tregs are human Tregs.

Activated cells can be characterized based on their forward scatter (FSC) profile. A higher FSC value, which is proportional to a cell's relative size indicates greater activation. The cell size (FSC-A) of Tregs produced according to the methods provided herein can be less than Tregs produced according to convention methods, e.g., Tregs that have been that have been stimulated for 6 days (about 144 hours) straight. (See, for instance, Example 3.) In some aspects, Tregs produced according to the methods provided herein have a relative cell size (FSC-A) that is less than 0.75 times that of Tregs that have been stimulated for 6 days (about 144 hours) straight. In some aspects, Tregs produced according to the methods provided herein have a relative cell size (FSC-A) that is less than 0.8 times that of Tregs that have been stimulated for 6 days (about 144 hours) straight. In some aspects, Tregs produced according to the methods provided herein have a relative cell size (FSC-A) that is less than 0.75 times that of Tregs that have been stimulated for 6 days (about 144 hours) straight.

Populations of Tregs are also provided herein. In some aspects, a population comprises at least 1×10³ cells, at least 1×10⁴ cells, at least 1×10⁵ cells, at least 1×10⁶ cells, at least 1×10⁷ cells, at least 1×10⁸ cells, at least 1×10⁹ cells, or at least 1×10¹⁰ cells. In some aspects, a population comprises at least 1×10³ Tregs, at least 1×10⁴ Tregs, at least 1×10⁵ Tregs, at least 1×10⁶ Tregs, at least 1×10⁷ Tregs, at least 1×10⁸ Tregs, at least 1×10⁹ Tregs, or at least 1×10¹⁰ Tregs.

In some aspects, at least 60% of the cells in a population of Tregs are Tregs. In some aspects, at least 70% of the cells in a population of Tregs are Tregs. In some aspects, at least 75% of the cells in a population of Tregs are Tregs. In some aspects, at least 80% of the cells in a population of Tregs are Tregs. In some aspects, at least 85% of the cells in a population of Tregs are Tregs. In some aspects, at least 90% of the cells in a population of Tregs are Tregs. In some aspects, at least 95% of the cells in a population of Tregs are Tregs. In some aspects, at least 96% of the cells in a population of Tregs are Tregs. In some aspects, at least 97% of the cells in a population of Tregs are Tregs. In some aspects, at least 98% of the cells in a population of Tregs are Tregs. In some aspects, at least 99% of the cells in a population of Tregs are Tregs.

In some aspects, less than 1% of the cells in a population of Tregs are effector T cells. In some aspects, less than 2% of the cells in a population of Tregs are effector T cells. In some aspects, less than 3% of the cells in a population of Tregs are effector T cells. In some aspects, less than 4% of the cells in a population of Tregs are effector T cells. In some aspects, less than 5% of the cells in a population of Tregs are effector T cells. In some aspects, less than 10% of the cells in a population of Tregs are effector T cells.

In some aspects, less than 1% of the cells in a population of Tregs are CD25⁻CD4⁺ or CD8⁺ T cells. In some aspects, less than 2% of the cells in a population of Tregs are CD25⁻CD4⁺ or CD8⁺ T cells. In some aspects, less than 3% of the cells in a population of Tregs are CD25⁻CD4⁺ or CD8⁺ T cells. In some aspects, less than 4% of the cells in a population of Tregs are CD25⁻CD4⁺ or CD8⁺ T cells. In some aspects, less than 5% of the cells in a population of Tregs are CD25⁻CD4⁺ or CD8⁺ T cells. In some aspects, less than 10% of the cells in a population of Tregs are CD25⁻CD4⁺ or CD8⁺ T cells. The proportion of cells in a population that are CD25⁻CD4⁺ or CD8⁺ T cells can be determined using flow cytometry.

In some aspects, a population of Tregs produced according to the methods provided has an average relative cell size (FSC-A) that is less than 0.75 times that of a population of Tregs that has been stimulated for 6 days (about 144 hours) straight. In some aspects, a population of Tregs produced according to the methods provided herein has an average relative cell size (FSC-A) that is less than 0.8 times that of a population of Tregs that has been stimulated for 6 days (about 144 hours) straight. In some aspects, a population of Tregs produced according to the methods provided herein has a relative cell size (FSC-A) that is less than 0.75 times that of a population of Tregs that has been stimulated for 6 days (about 144 hours) straight.

The proportion of Helios⁺Foxp3⁺ Tregs in a Treg population produced according to the methods provided herein can be less than Treg populations produced according to convention methods, e.g., Treg populations that have been stimulated for 6 days (about 144 hours) straight. (See, for instance, Example 7.) In some aspects, a Treg population produced according to the methods provided herein can have at least 1.5 times the percentage of Helios +Foxp3⁺CD4⁺ T cells as a Treg population that has been stimulated for 6 days (about 144 hours) straight. In some aspects, a Treg population produced according to the methods provided herein can have at least 2 times the percentage of Helios⁺Foxp3⁺CD4⁺ T cells as a Treg population that has been stimulated for 6 days (about 144 hours) straight. In some aspects, a Treg population produced according to the methods provided herein can have 1.5 to 3 times the percentage of Helios⁺Foxp3⁺CD4⁺ T cells as a Treg population that has been stimulated for 6 days (about 144 hours) straight. In some aspects, a Treg population produced according to the methods provided herein can have 1.5 to 2.5 times the percentage of Helios⁺Foxp3⁺CD4⁺ T cells as a Treg population that has been stimulated for 6 days (about 144 hours) straight. The proportion of Helios⁺Foxp3⁺ Tregs in a Treg population can be determined using flow cytometry.

In some aspects, at least 50% of cells in a population of Tregs produced by the methods described herein express Helios. In some aspects, at least 60% of cells in a population of Tregs produced by the methods described herein express Helios. In some aspects, at least 70% of cells in a population of Tregs produced by the methods described herein express Helios. In some aspects, at least 75% of cells in a population of Tregs produced by the methods described herein express Helios. In some aspects, at least 80% of cells in a population of Tregs produced by the methods described herein express Helios. In some aspects, at least 85% of cells in a population of Tregs produced by the methods described herein express Helios. In some aspects, at least 90% of cells in a population of Tregs produced by the methods described herein express Helios. In some aspects, at least 95% of cells in a population of Tregs produced by the methods described herein express Helios. In some aspects, at least 96% of cells in a population of Tregs produced by the methods described herein express Helios. In some aspects, at least 97% of cells in a population of Tregs produced by the methods described herein express Helios. In some aspects, at least 98% of cells in a population of Tregs produced by the methods described herein express Helios. The percentage of cells in a population of Tregs that express Helios can be determined using flow cytometry.

In some aspects, the proportion of Helios-expressing Tregs does not decrease by more than 50% when Tregs are expanded (and optionally genetically engineered) according to the methods provided herein. In some aspects, the proportion of Helios-expressing Tregs does not decrease by more than 40% when Tregs are expanded (and optionally genetically engineered) according to the methods provided herein. In some aspects, the proportion of Helios-expressing Tregs does not decrease by more than 30% when Tregs are expanded (and optionally genetically engineered) according to the methods provided herein. In some aspects, the proportion of Helios-expressing Tregs does not decrease by more than 25% when Tregs are expanded (and optionally genetically engineered) according to the methods provided herein.

In some aspects, at least 60% of cells in a population of Tregs produced by the methods described herein express FOXP3. In some aspects, at least 70% of cells in a population of Tregs produced by the methods described herein express FOXP3. In some aspects, at least 75% of cells in a population of Tregs produced by the methods described herein express FOXP3. In some aspects, at least 80% of cells in a population of Tregs produced by the methods described herein express FOXP3. In some aspects, at least 85% of cells in a population of Tregs produced by the methods described herein express FOXP3. In some aspects, at least 90% of cells in a population of Tregs produced by the methods described herein express FOXP3. In some aspects, at least 95% of cells in a population of Tregs produced by the methods described herein express FOXP3. In some aspects, at least 96% of cells in a population of Tregs produced by the methods described herein express FOXP3. In some aspects, at least 97% of cells in a population of Tregs produced by the methods described herein express FOXP3. In some aspects, at least 98% of cells in a population of Tregs produced by the methods described herein express FOXP3. The percentage of cells in a population of Tregs that express FOXP3 can be determined using flow cytometry.

In some aspects, at least 50% of cells in a population of Tregs produced by the methods described herein express FOXP3 and Helios. In some aspects, at least 60% of cells in a population of Tregs produced by the methods described herein express FOXP3 and Helios. In some aspects, at least 70% of cells in a population of Tregs produced by the methods described herein express FOXP3 and Helios. In some aspects, at least 75% of cells in a population of Tregs produced by the methods described herein express FOXP3 and Helios. In some aspects, at least 80% of cells in a population of Tregs produced by the methods described herein express FOXP3 and Helios. In some aspects, at least 85% of cells in a population of Tregs produced by the methods described herein express FOXP3 and Helios. In some aspects, at least 90% of cells in a population of Tregs produced by the methods described herein express FOXP3 and Helios. In some aspects, at least 95% of cells in a population of Tregs produced by the methods described herein express FOXP3 and Helios. In some aspects, at least 96% of cells in a population of Tregs produced by the methods described herein express FOXP3 and Helios. In some aspects, at least 97% of cells in a population of Tregs produced by the methods described herein express FOXP3 and Helios. In some aspects, at least 98% of cells in a population of Tregs produced by the methods described herein express FOXP3 and Helios. The percentage of cells in a population of Tregs that express FOXP3 and Helios can be determined using flow cytometry.

As demonstrated herein, a Treg population produced according to the methods provided herein can have an increased ability to suppress proliferation of effector T cells (Teffs) as compared Treg populations produced according to convention methods, e.g., Treg populations that have been stimulated for 6 days (about 144 hours) straight. (See, for instance, Example 8.)

The ability of a Treg population to suppress proliferation of Teffs can be determined, for example, using an in vitro suppression assay as provided in Example 1 herein. The ability of a Treg population to suppress proliferation of Teffs can be detected in vitro.

In one aspect provided herein, the ability of Treg population to suppress proliferation of Teffs is determined using the following assay (i) stimulating Tregs that have been rested overnight in Treg media containing 10 μl/mL of ImmunoCult CD3/28/2 tetramer (StemCell Technologies, Cat #10970) and IL-2 (300 units/mL), (ii) washing Tregs to remove the tetramer and IL-2, (iii) resuspending the Tregs in Treg media, (iv) mixing the Tregs with cell trace violate (CTV) labeled PBMCs (e.g., at a ratio of 1:1 to 1:16), (v) adding 0.1 mL of ImmunoCult CD3/28/2 tetramer (3 μl/mL) for 4 days (about 96 hours), and (vi) staining the cells for CD3, CD4, and CD8. A lower CTV signal from CD4+ or CD8+ T effector cells in the presence of the Tregs as compared to the absence of the Tregs indicates that the Tregs suppress proliferation of the Teffs.

In some aspects, a Treg population produced according to the methods provided herein can have at least twice the ability to suppress proliferation of Teffs as a Treg population that has been stimulated for 6 days (about 144 hours) straight. In some aspects, a Treg population produced according to the methods provided herein can have at least three times the ability to suppress proliferation of Teffs as a Treg population that has been stimulated for 6 days (about 144 hours) straight. In some aspects, a Treg population produced according to the methods provided herein can have at least four times the ability to suppress proliferation of Teffs as a Treg population that has been stimulated for 6 days (about 144 hours) straight. In some aspects, a Treg population produced according to the methods provided herein can have at least five times the ability to suppress proliferation of Teffs as a Treg population that has been stimulated for 6 days (about 144 hours) straight. In some aspects, a Treg population produced according to the methods provided herein can have at least six times the ability to suppress proliferation of Teffs as a Treg population that has been stimulated for 6 days (about 144 hours) straight. In some aspects, a Treg population produced according to the methods provided herein can have at least seven times the ability to suppress proliferation of Teffs as a Treg population that has been stimulated for 6 days (about 144 hours) straight. In some aspects, a Treg population produced according to the methods provided herein can have at least eight times the ability to suppress proliferation of Teffs as a Treg population that has been stimulated for 6 days (about 144 hours) straight.

In some aspects, a Treg population produced according to the methods provided herein can have about 2 to about 10 times the ability to suppress proliferation of Teffs as a Treg population that has been stimulated for 6 days (about 144 hours) straight. In some aspects, a Treg population produced according to the methods provided herein can have about 4 to about 10 times the ability to suppress proliferation of Teffs as a Treg population that has been stimulated for 6 days (about 144 hours) straight. In some aspects, a Treg population produced according to the methods provided herein can have about 6 to about 10 times the ability to suppress proliferation of Teffs as a Treg population that has been stimulated for 6 days (about 144 hours) straight.

In some aspects, the steps of a method (e.g., all stimulating step, all rest steps, and all editing steps, when present) described herein are completed in less than 30 days, or less than 29 days, or less than 28 days, or less than 27 days, or less than 26 days, or less than 25 days, or less than 24 days, or less than 23 days, or less than 22 days, or less than 21 days, or less than 20 days, or less than 19 days, or less than 18 days, or less than 17 days, or less than 16 days, or less than 14 days, or less than 14 days, or between 14 and 30 days, or between 14 and 25 days, or between 15 and 28 days, or between 15 and 25 days.

### Stimulation of Tregs

As provided herein, methods of expanding a population of Tregs can comprise a stimulating step wherein a population of Tregs is cultured in presence of a stimulatory agent. A stimulating step can result in the production of a stimulated population of Tregs.

In some aspects of the methods provided herein, culturing a population of Tregs in the presence of a stimulatory agent results in at least 40% of T cell receptors and/or co-stimulatory molecules (e.g., CD28 or GITR) on the Tregs in the population being activated. In some aspects of the methods provided herein, culturing a population of Tregs in the presence of a stimulatory agent results in at least 50% of T cell receptors and/or co-stimulatory molecules (e.g., CD28 or GITR) on the Tregs in the population being activated. In some aspects of the methods provided herein, culturing a population of Tregs in the presence of a stimulatory agent results in at least 60% of T cell receptors and/or co-stimulatory molecules (e.g., CD28 or GITR) on the Tregs in the population being activated. In some aspects of the methods provided herein, culturing a population of Tregs in the presence of a stimulatory agent results in at least 70% of T cell receptors and/or co-stimulatory molecules (e.g., CD28 or GITR) on the Tregs in the population being activated. In some aspects of the methods provided herein, culturing a population of Tregs in the presence of a stimulatory agent results in at least 80% of T cell receptors and/or co-stimulatory molecules (e.g., CD28 or GITR) on the Tregs in the population being activated. In some aspects of the methods provided herein, culturing a population of Tregs in the presence of a stimulatory agent results in at least 90% of T cell receptors and/or co-stimulatory molecules (e.g., CD28 or GITR) on the Tregs in the population being activated. In some aspects of the methods provided herein, culturing a population of Tregs in the presence of a stimulatory agent results in at least 95% of T cell receptors and/or co-stimulatory molecules (e.g., CD28 or GITR) on the Tregs in the population being activated. In some aspects of the methods provided herein, culturing a population of Tregs in the presence of a stimulatory agent results in at least 96% of T cell receptors and/or co-stimulatory molecules (e.g., CD28 or GITR) on the Tregs in the population being activated. In some aspects of the methods provided herein, culturing a population of Tregs in the presence of a stimulatory agent results in at least 97% of T cell receptors and/or co-stimulatory molecules (e.g., CD28 or GITR) on the Tregs in the population being activated. In some aspects of the methods provided herein, culturing a population of Tregs in the presence of a stimulatory agent results in at least 98% of T cell receptors and/or co-stimulatory molecules (e.g., CD28 or GITR) on the Tregs in the population being activated. In some aspects of the methods provided herein, culturing a population of Tregs in the presence of a stimulatory agent results in at least 99% of T cell receptors and/or co-stimulatory molecules (e.g., CD28 or GITR) on the Tregs in the population being activated.

During an individual stimulating step, Tregs are continuously cultured in the presence of a stimulatory agent. In some aspects, an individual stimulating step involves a single addition of a stimulating agent to the Treg culture (at the beginning of the stimulating step), such that the concentration of the stimulating agent can vary (e.g., decrease) throughout the stimulating step. In some aspects, an individual stimulating step involves repeated addition of a stimulating agent to the Treg culture to maintain a desired concentration of stimulating agent.

Some methods provided herein begin with a stimulating step. A stimulating step can also occur after a resting step and/or can precede a resting step. In some aspects, a stimulating step occurs between two resting steps.

A stimulatory agent can be an antigen non-specific stimulator (such as an anti-CD3 antibody) or an antigen-specific stimulator, such as an MHC-peptide multimer. In some aspects, a stimulatory agent activates a T cell receptor on a Treg, e.g, by binding to a TCR. In some aspects, a stimulatory agent activates CD28 on a Treg, e.g., by binding to CD28. In some aspects, a stimulatory agent activates a T cell receptor and CD28 on a Treg, e.g., by binding to a TCR and to CD28. In some aspects, a stimulatory agent is a mitogen such as PHA or ConA.

In some aspects, a stimulatory agent is an antibody or antigen-binding fragment

thereof. In some aspects, a stimulatory agent is an antibody complex. In some aspects, a stimulatory agent is a tetrameric antibody complex. In some aspects, the tetrameric antibody complex specifically binds to CD3, CD28, and/or CD2. In some aspects, the tetrameric antibody complex specifically binds to CD3, CD28, and CD2. An exemplary tetrameric antibody complex specifically binds to CD3, CD28, and CD2 is available as ImmunoCult CD3/28/2 tetramer from StemCell Technologies, Cat #10970.

In some aspects, the tetrameric antibody complex is present at a concentration of at least 0.5 ng/ml.

In some aspects, a stimulatory agent is an anti-CD3 antibody or antigen-binding fragment thereof. In some aspects, an anti-CD3 antibody or antigen-binding fragment thereof is OKT3. Such a stimulatory agent can be present at a concentration of at least 0.5 ng/ml.

The term “anti-CD3 antibody” refers to an antibody, e.g., a monoclonal antibody and including human, humanized, chimeric, and murine antibodies which are directed against the CD3 receptor in the T cell antigen receptor of mature T cells. Anti-CD3 antibodies include OKT-3, also known as muromonab. Anti-CD3 antibodies also include the UHCT1 clone, also known as T3 and CD3c. Other anti-CD3 antibodies include, for example, otelixizumab, teplizumab, and visilizumab.

The “OKT-3” antibody (also referred to herein as “OKT3”) is commercially available, for example, as OKT-3 30 ng/mL, MACS GMP CD3 pure, from Miltenyi Biotech, Inc., San Diego, Calif., USA). The amino acid sequences of the heavy and light chains of muromonab are given in SEQ ID NO:1 and SEQ ID NO:2, respectively.

(SEQ ID NO: 1)
QVQLQQSGAELARPGASVKMSCKASGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSSAKTTAPSVYPLAPVCGGTTGSSVTLGCL
VKGYFPEPVTLTWNSGSLSSGVHTFPAVLQSDLYTLSSSVTVTSSTWPS
QSITCNVAHPASSTKVDKKIEPRPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
(SEQ ID NO: 2)
QIVLTQSPAIMSASPGEKVTMTCSASSSVSYMNWYQQKSGTSPKRWIYD
TSKLASGVPAHFRGSGSGTSYSLTISGMEAEDAATYYCQQWSSNPFTFG
SGTKLEINRADTAPTVSIFPPSSEQLTSGGASVVCFLNNFYPKDINVKW
KIDGSERQNGVLNSWTDQDSKDSTYSMSSTLTLTKDEYERHNSYTCEAT
HKTSTSPIVKSFNRNEC

In some aspects, a stimulatory agent is an anti-CD28 antibody or antigen-binding fragment thereof. Such a stimulatory agent can be present at a concentration of at least 0.5 ng/ml.

A stimulatory agent can be on a substrate, such as a cell or bead. Beads can be plastic, glass, or any other suitable material, typically in the 1-20 micron range. In some aspects, the beads are paramagnetic beads. In some aspects, the beads are supermagnetic beads. Thus, for example, a stimulatory agent can be an anti-CD3-coated supermagnetic bead and/or an anti-CD28-coated supermagnetic bead. Such a stimulatory agent can be present, e.g., at a concentration of about 5 μg/ml or about 10 μg/ml. Such a stimulatory agent can be present, e.g., at a concentration of about 2.5 μg/ml to about 15 μg/ml.

Cells suitable for use as substrates include antigen presenting cells (APCs) and artificial antigen-presenting cells (aAPCs).

In some aspects, a stimulatory agent is an antigen presenting cell (APC) or an artificial antigen presenting cell (aAPC). An aAPC can be an irradiated aAPC.

In some aspects, the stimulatory agent is not on a bead.

In some aspects, a stimulatory agent is tetrameric antibody complex specifically binds to CD3, CD28, and/or CD2, an anti-CD3 antibody or antigen-binding fragment thereof, an anti-CD28 antibody or antigen-binding fragment thereof, and/or an antigen presenting cell or artificial antigen presenting cell.

In some aspects, a stimulatory agent is present in a concentration sufficient to increase Treg proliferation, for example, by at least 1.5 fold within 72 hours. In some aspects, a stimulatory agent is present in a concentration sufficient to increase Treg proliferation, for example, by at least 2 fold within 72 hours. In some aspects, a stimulatory agent is present in a concentration sufficient to increase Treg proliferation, for example, by at least 3 fold within 72 hours. Treg proliferation can be measured using a luminescent cell viability assay such as Cell Titer Glo®, which determines the number of viable, metabolically active cells in culture based on quantitation of ATP.

In some aspects, a stimulatory agent is present in a concentration sufficient to increase a cell's forward scatter (FSC) profile, a commonly used indicator of the activation state of T cells cultured in vitro.

As provided herein, a method of expanding a population of Tregs can comprise multiple stimulating steps. In such aspects, the stimulating steps can use the same stimulatory agent or the stimulating steps can use different stimulatory agents. Where multiple stimulating steps use the same stimulatory agent, the stimulating agents can use the same concentration of the stimulatory agent or can use different concentrations of the stimulatory agent. Stimulatory agents can be used in combination such that a single stimulating step can involve the use of two stimulatory agents (simultaneously and/or consecutively). Thus, in some aspects, a stimulatory agent is a CD3- and CD28-coated supermagnetic bead.

In some aspects, a method provided herein comprises two stimulating steps (separated by a resting step). In some aspects, a method provided herein comprises at least two stimulating steps (each separated by a resting step). In some aspects, a method provided herein comprises at least three stimulating steps (each separated by a resting step).

In some aspects, a stimulating step is at least 1 day (about 24 hours). In some aspects, a stimulating step is at least 2 days (about 48 hours). In some aspects, a stimulating step is at least 3 days (about 72 hours). In some aspects, a stimulating step is at least 4 days (about 96 hours).

In some aspects, a stimulating step is about one days (about 24 hours). In some aspects, a stimulating step is about two days (about 48 hours). In some aspects, a stimulating step is about three days (about 72 hours). In some aspects, a stimulating step is about four days (about 96 hours).

In some aspects, a stimulating step is about 1 day (about 24 hours) to about 5 days (about 120 hours). In some aspects, a stimulating step is about 1 day (about 24 hours) to about 4 days (about 96 hours). In some aspects, a stimulating step is about 1 day (about 24 hours) to about 3 days (about 72 hours). In some aspects, a stimulating step is about 2 days (about 48 hours) to about 5 days (about 120 hours). In some aspects, a stimulating step is about 2 days (about 48 hours) to about 4 days (about 96 hours). In some aspects, a stimulating step is about 3 days (about 72 hours) to about 4 days (about 96 hours).

In some days a stimulating step does not exceed 6 days (about 144 hours) or does not exceed 5 days (about 120 hours).

In some aspects, a method provided herein comprises at least two (e.g., two) stimulating steps (each separated by a resting step), wherein the first stimulating step is longer than the second stimulating step. In some aspects, the first stimulating step is longer than the second stimulating step by at least about 12 hours, at least about 24 hours, at least about 36 hours, at least about 48 hours, at least about 60 hours, or at least about 72 hours. The first stimulating step can be, for example, about 1 day (about 24 hours) to about 4 days (about 96 hours), and the second stimulating step can be, for example, about 1 day (about 24 hours) to about 3 days (about 72 hours). In some aspects, the first stimulating step is about 3 days (about 72 hours) and the second stimulating step is about 2 days (about 48 hours).

In some aspects, a method provided herein comprises at least one stimulating step prior to genetically engineering Tregs. In some aspects, a method provided herein comprises at least two stimulating steps (each separated by a resting step) prior to genetically engineering Tregs.

In some aspects, a method provided herein comprises at least one stimulating step after genetically engineering Tregs.

### Resting Tregs

As provided herein, methods of expanding a population of Tregs can comprise a resting step wherein a population of Tregs is cultured in the absence of a stimulatory agent. A resting step can result in the production of a rested population of Tregs. As used herein, resting refers to the absence of a stimulatory agent (and not, e.g., to the absence of cell growth, division, expansion, or any other activity of a Treg).

In some aspects of the methods provided herein, a significant portion of the Tregs in a Treg population cultured in the absence of a stimulatory agent do not comprise active T cell receptors and/or co-stimulatory molecules, such as CD28 or GITR. In some aspects of the methods provided herein, culturing a population of Tregs in the absence of a stimulatory agent results in no more than 20% of T cell receptors and/or co-stimulatory molecules (e.g., CD28 or GITR) on the Tregs in the population being activated. In some aspects of the methods provided herein, culturing a population of Tregs in the absence of a stimulatory agent results in no more than 15% of T cell receptors and/or co-stimulatory molecules (e.g., CD28 or GITR) on the Tregs in the population being activated. In some aspects of the methods provided herein, culturing a population of Tregs in the absence of a stimulatory agent results in no more than 10% of T cell receptors and/or co-stimulatory molecules (e.g., CD28 or GITR) on the Tregs in the population being activated. In some aspects of the methods provided herein, culturing a population of Tregs in the absence of a stimulatory agent results in no more than 5% of T cell receptors and/or co-stimulatory molecules (e.g., CD28 or GITR) on the Tregs in the population being activated.

Those of ordinary skill in the art will be aware of methods for determining whether a a population of Tregs is cultured in the absence of a stimulatory agent. For example, removal/absence of stimulating agent may be quantified using methods known to those of ordinary skill in the art, for example, by determining changes in cell size and/or granularity, by determining drownregulation of Treg activation markers, and/or by downregulation of immunosuppressive cytokine production.

In some aspects, the absence of a stimulating agent is determined by determining changes in cell size and/and granularity. In some aspects, the changes in cell size and/and granularity are measured using a flow cytometer (i.e., F SC/SSC measurements). As noted herein, activated cells can be characterized based on their forward scatter (FSC) profile. In some aspects, in the absence of a stimulating agent, the cell size (F SC-A) of the population of Tregs is about 40% less, or about 50% less, or about 60% less, or about 70% less, or about 75% less, or about 80% less, or about 90% less, or less as compared to the cell size (F SC-A) of the population of Tregs prior to removal of the stimulating agent. In some aspects, the cell size (FSC-A) of the population of Tregs in the absence of a stimulating agent is determined at about 12 hours, or about 18 hours, or about 24 hours, or about 36 hours, or about 48 hours, or about 72 hours following removal of the stimulating agent. In some aspects, the cell size (FSC-A) of the population of Tregs in prior to removal of the stimulating agent is determined at about 2 hours, about 1 hour, about 30 minutes, about 15 minutes, or less, prior to removal of the stimulating agent.

Other non-limiting examples for determining the absence of a stimulating agent include determining the drownregulation of Treg activation markers (e.g., Foxp3, Helios, CTLA4, CD25, CD69, HLA-DR) and/or determining the downregulation of immunosuppressive cytokine production (e.g. IL-10, TGFb). In some aspects, in the absence of a stimulating agent, the expression of the Treg activation markers and/or immuosuppressive cystokine production is about 40% less, or about 50% less, or about 60% less, or about 70% less, or about 75% less, or about 80% less, or about 90% less, or less as compared to the expression of the Treg activation markers and/or immuosuppressive cystokine production of the population of Tregs prior to removal of the stimulating agent. In some aspects, the expression of the Treg activation markers and/or immuosuppressive cystokine production of the population of Tregs in the absence of a stimulating agent is determined at about 12 hours, or about 18 hours, or about 24 hours, or about 36 hours, or about 48 hours, or about 72 hours following removal of the stimulating agent. In some aspects, the expression of the Treg activation markers and/or immuosuppressive cystokine production of the population of Tregs in prior to removal of the stimulating agent is determined at about 2 hours, about 1 hour, about 30 minutes, about 15 minutes, or less, prior to removal of the stimulating agent.

A resting step can be initiated by removing stimulatory agent from a Treg culture. The stimulatory agent can be removed for example, by a washing step and/or a centrifugation step. As demonstrated herein, removal of stimulation for a resting step, e.g., after 2 days (about 48 hours), or 3 days (about 72 hours), or 4 days (about 96 hours), or more of stimulation, can result in improved Treg growth.

In some aspects, the concentration of a stimulating agent is reduced by at least 25% in a resting step as compared to the concentration of stimulating agent present in a stimulating step. In some aspects, the concentration of a stimulating agent is reduced by at least 50% in a resting step as compared to the concentration of stimulating agent present in a stimulating step. In some aspects, the concentration of a stimulating agent is reduced by at least 60% in a resting step as compared to the concentration of stimulating agent present in a stimulating step. In some aspects, the concentration of a stimulating agent is reduced by at least 70% in a resting step as compared to the concentration of stimulating agent present in a stimulating step. In some aspects, the concentration of a stimulating agent is reduced by at least 80% in a resting step as compared to the concentration of stimulating agent present in a stimulating step. In some aspects, the concentration of a stimulating agent is reduced by at least 90% in a resting step as compared to the concentration of stimulating agent present in a stimulating step. In some aspects, the concentration of a stimulating agent is reduced by at least 91% in a resting step as compared to the concentration of stimulating agent present in a stimulating step. In some aspects, the concentration of a stimulating agent is reduced by at least 92% in a resting step as compared to the concentration of stimulating agent present in a stimulating step. In some aspects, the concentration of a stimulating agent is reduced by at least 93% in a resting step as compared to the concentration of stimulating agent present in a stimulating step. In some aspects, the concentration of a stimulating agent is reduced by at least 94% in a resting step as compared to the concentration of stimulating agent present in a stimulating step. In some aspects, the concentration of a stimulating agent is reduced by at least 95% in a resting step as compared to the concentration of stimulating agent present in a stimulating step. In some aspects, the concentration of a stimulating agent is reduced by at least 96% in a resting step as compared to the concentration of stimulating agent present in a stimulating step. In some aspects, the concentration of a stimulating agent is reduced by at least 97% in a resting step as compared to the concentration of stimulating agent present in a stimulating step. In some aspects, the concentration of a stimulating agent is reduced by at least 98% in a resting step as compared to the concentration of stimulating agent present in a stimulating step. In some aspects, the concentration of a stimulating agent is reduced by at least 99% in a resting step as compared to the concentration of stimulating agent present in a stimulating step. In some aspects, there is no stimulatory agent present in a resting step, i.e., the Tregs are cultured without any stimulatory agent. Some methods provided herein begin with a resting step. A resting step can also occur after a stimulating step and/or can precede a stimulating step. In some aspects, a resting step occurs between two stimulating steps.

In some aspects, a method provided herein comprises at least one resting step. In some aspects, a method provided herein comprises at least two resting steps.

In some aspects, a resting step is at least 1 day (about 24 hours).

In some aspects, a resting step is at least 2 days (about 48 hours).

In some aspects, a resting step is about 3 days (about 72 hours).

In some aspects, a resting step is about 4 days (about 96 hours).

In some aspects, a resting step is about 5 days (about 120 hours).

In some aspects, a resting step is about 1 day (about 24 hours) to about 5 days (about 120 hours). In some aspects, a resting step is about 1 day (about 24 hours) to about 4 days (about 96 hours). In some aspects, a resting step is about 1 day (about 24 hours) to about 3 days (about 72 hours). In some aspects, a resting step is about 2 days (about 48 hours) to about 4 days (about 96 hours). In some aspects, a resting step is about 2 days (about 48 hours) to about 5 days (about 120 hours). In some aspects, a resting step is about 3 days (about 72 hours) to about 4 days (about 96 hours).

In some aspects, a method provided herein comprises at least one resting step prior to genetically engineering Tregs. As used herein a resting step “prior to” genetically engineered does not require that the resting step occurs immediately prior to the genetic engineering. Thus, a method that comprises a stimulating step, then a resting step, then a stimulating step, and then genetic engineering is a method that comprises a resting step prior to genetic engineered.

In some aspects, a method provided herein comprises at least one resting step after genetically engineering Tregs.

In some aspects, a method provided herein comprises a resting step after Tregs are genetically engineered. In some aspects, Tregs are genetically engineered and then rested without a stimulating step between the genetic engineering and the resting step.

In some aspects, a method provided herein comprises (i) at least one resting step before Tregs are genetically engineered and (ii) at least one resting step after the Tregs are genetically engineered, e.g., before the Tregs are stimulated again.

### Expansion of Tregs

As provided herein, methods of expanding Tregs can include alternating periods

of stimulating the Tregs and resting the Tregs or “discontinuous stimulation.” Accordingly, in some aspects, a method provided herein comprises at least one stimulating step and at least one resting step. In some aspects, a method provided herein comprises at least two stimulating steps separated by a resting step. In some aspects, a method provided herein comprises at least three stimulating steps, each separated by a resting step. In some aspects, the the Tregs can be genetically engineered (as discussed elsewhere herein), e.g., during a resting step (i.e., the Tregs can be genetically engineered in the absence of a stimulating agent). In some aspects, a method provided herein does not comprise genetic engineering.

In some aspects, a method provided herein comprises a first stimulating step, followed be a first resting step, followed be a second stimulating step, followed by a second resting step. In some aspects, the Tregs are harvested after a second resting step.

In some aspects, a method provided herein comprises a first stimulating step, followed be a first resting step, followed be a second stimulating step, followed by a second resting step, followed by a third stimulating step, followed by a third resting step. In some aspects, the Tregs are harvested after a third resting step.

In some aspects, a method provided herein comprises a first stimulating step, followed be a first resting step, followed be a second stimulating step, followed by a second resting step during which the Tregs are genetically engineered, followed by a third stimulating step, followed by a third resting step. In some aspects, the Tregs are harvested after a third resting step.

Non-limiting examples of the times for the stimulating steps and resting steps are described herein. In a non-limiting aspect, the first stimulating step can be about 2 days (about 48 hours) to about 5 days (about 120 hours) (e.g., about 3 days (about 72 hours)); the first resting step can about 2 days (about 48 hours) to about 5 days (about 120 hours) (e.g., about 3 days (about 72 hours)); the second stimulating step can be about 2 days (about 48 hours) to about 5 days (about 120 hours) (e.g., about 2 days (about 48 hours)); the second resting step can be about 2 days (about 48 hours) to about 5 days (about 120 hours) (e.g., about 3 days (about 72 hours)); the third stimulating step can be about 2 days (about 48 hours) to about 5 days (about 72 hours) (e.g., about 2 days (about 48 hours)); and the third resting step can be about 2 days (about 48 hours) to about 5 days (about 120 hours) (e.g., about 2 days (about 48 hours)). Non-limiting examples of time periods for the resting and stimulating steps are provided in Table A.

TABLE A
Exemplary time periods for resting and stimulating steps

| | | | | Optional | Optional |
|---|---|---|---|---|---|
| First | First | Second | Second | Third | Third |
| Stimulating | Resting | Stimulating | Resting | Stimulating | Resting |
| Step | Step | step | Step | Step | Step |
| (days) | (days) | (days) | (days) | (days) | (days) |
| 1-5 | 1-5 | 1-5 | 1-5 | 1-5 | 1-5 |
| 1-5 | 1-4 | 1-5 | 1-4 | 1-5 | 1-4 |
| 1-5 | 1-3 | 1-5 | 1-3 | 1-5 | 1-3 |
| 1-5 | 2-5 | 1-5 | 2-5 | 1-5 | 2-5 |
| 1-5 | 2-4 | 1-5 | 2-4 | 1-5 | 2-4 |
| 1-5 | 2-3 | 1-5 | 2-3 | 1-5 | 2-3 |
| 1-4 | 1-5 | 1-4 | 1-5 | 1-4 | 1-5 |
| 1-4 | 1-4 | 1-4 | 1-4 | 1-4 | 1-4 |
| 1-4 | 1-3 | 1-4 | 1-3 | 1-4 | 1-3 |
| 1-4 | 2-5 | 1-4 | 2-5 | 1-4 | 2-5 |
| 1-4 | 2-4 | 1-4 | 2-4 | 1-4 | 2-4 |
| 1-4 | 2-3 | 1-4 | 2-3 | 1-4 | 2-3 |
| 1-3 | 1-5 | 1-3 | 1-5 | 1-3 | 1-5 |
| 1-3 | 1-4 | 1-3 | 1-4 | 1-3 | 1-4 |
| 1-3 | 1-3 | 1-3 | 1-3 | 1-3 | 1-3 |
| 1-3 | 2-5 | 1-3 | 2-5 | 1-3 | 2-5 |
| 1-3 | 2-4 | 1-3 | 2-4 | 1-3 | 2-4 |
| 1-3 | 2-3 | 1-3 | 2-3 | 1-3 | 2-3 |
| 2-5 | 1-5 | 2-5 | 1-5 | 2-5 | 1-5 |
| 2-5 | 1-4 | 2-5 | 1-4 | 2-5 | 1-4 |
| 2-5 | 1-3 | 2-5 | 1-3 | 2-5 | 1-3 |
| 2-5 | 2-5 | 2-5 | 2-5 | 2-5 | 2-5 |
| 2-5 | 2-4 | 2-5 | 2-4 | 2-5 | 2-4 |
| 2-5 | 2-3 | 2-5 | 2-3 | 2-5 | 2-3 |
| 2-4 | 1-5 | 2-4 | 1-5 | 2-4 | 1-5 |
| 2-4 | 1-4 | 2-4 | 1-4 | 2-4 | 1-4 |
| 2-4 | 1-3 | 2-4 | 1-3 | 2-4 | 1-3 |
| 2-4 | 2-5 | 2-4 | 2-5 | 2-4 | 2-5 |
| 2-4 | 2-4 | 2-4 | 2-4 | 2-4 | 2-4 |
| 2-4 | 2-3 | 2-4 | 2-3 | 2-4 | 2-3 |
| 2-3 | 1-5 | 2-3 | 1-5 | 2-3 | 1-5 |
| 2-3 | 1-4 | 2-3 | 1-4 | 2-3 | 1-4 |
| 2-3 | 1-3 | 2-3 | 1-3 | 2-3 | 1-3 |
| 2-3 | 2-5 | 2-3 | 2-5 | 2-3 | 2-5 |
| 2-3 | 2-4 | 2-3 | 2-4 | 2-3 | 2-4 |
| 2-3 | 2-3 | 2-3 | 2-3 | 2-3 | 2-3 |

In some aspects, the Tregs are cryopreserved.

As provided herein, Tregs can be cultured in a media suitable for Tregs. Exemplary T cell media can comprise, for example (i) X-VIVO 15 T Cell Expansion Medium (Lonza, Cat #04-418Q) supplemented with 10% human inactivated serum, (ii) RPMI 1640 media supplemented with 5 mM HEPES, 2 mM L-gluamine, 50 mg/ml penicillin, 50 mg/ml streptomycin, 5 mM nonessential amino acids, 5 mM sodium pyruvate, and 10% FBS, or (iii) 10% heat-inactivated fetal bovine serum (Biosource International), nonessential amino acids, 0.5 mM sodium pyruvate, 5 mM Hepes, 1 mM glutaMax I, and 55 μM β-mercaptoethanol in DMEM base.

The Treg culturing media can further comprise a cytokine, such as an interleukin, such as interleukin-2 (IL-2), interleukin-15 (IL-15), and/or interleukin-7 (IL-7).

The term “IL-2” (or “IL2”) refers to the cytokine and T cell growth factor known as interleukin-2, and includes all forms of IL-2, including human and mammalian forms, forms with conservative amino acid substitutions, glycoforms, biosimilars, and variants thereof. IL-2 is described, e.g., in Nelson, J. Immunol. 2004, 172, 3983-88 and Malek, Annu. Rev. Immunol. 2008, 26, 453-79, the disclosures of which are incorporated herein by reference in their entireties. The term IL-2 encompasses human, recombinant forms of IL-2, such as aldesleukin (PROLEUKIN, available commercially from multiple suppliers in 22 million IU per single use vials), as well as the form of recombinant IL-2 commercially supplied by CellGenix, Inc., Portsmouth, N.H., USA (CELLGRO GMP) or ProSpec-Tany TechnoGene Ltd., East Brunswick, N.J., USA (Cat. No. CYT-209-b) and other commercial equivalents from other vendors. Aldesleukin (des-alanyl-1, serine-125 human IL-2) is a nonglycosylated human recombinant form of IL-2 with a molecular weight of approximately 15 kDa. The term IL-2 also encompasses pegylated forms of IL-2, including the pegylated IL-2 prodrug NKTR-214, available from Nektar Therapeutics, South San Francisco, Calif., USA. NKTR-214 and pegylated IL-2 suitable for use in the invention is described in U.S. Patent Application Publication No. US 2014/0328791 A1 and International Patent Application Publication No. WO 2012/065086 A1, the disclosures of which are incorporated herein by reference in their entireties. Alternative forms of conjugated IL-2 suitable for use in the invention are described in U.S. Pat. Nos. 4,766,106, 5,206,344, 5,089,261 and 4,902,502, the disclosures of which are incorporated herein by reference in their entireties. Formulations of IL-2 suitable for use in the invention are described in U.S. Pat. No. 6,706,289, the disclosure of which is incorporated herein by reference in its entirety. The human IL2 gene is identified by NCBI Gene ID 3558. An exemplary nucleotide sequence for a human IL2 gene is the NCBI Reference Sequence: NG_016779.1.

The amino acid sequence of a recombinant human IL-2 suitable for use herein is:

(SEQ ID NO: 3)
MAPTSSSTKKTQLQLEHLLLDLQMILNGINNYKNPKLTRMLTFKFYMPK
KATELKHLQCLEEELKPLEEVLNLAQSKNFHLRPRDLISNINVIVLELK
GSETTFMCEYADETATIVEFLNRWITFCQSIISTLT.

Aldesleukin (des-alanyl-1, serine-125 human IL-2) is a nonglycosylated human recombinant form of IL-2 with a molecular weight of approximately 15 kDa. The amino acid sequence of aldesleukin suitable for use in the methods provided herein is:

(SEQ ID NO: 4)
PTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGS
ETTFMCEYADETATIVEFLNRWITFSQSIISTLT.

It has been reported that Interleukin-2 (IL-2) is a type of cytokine signaling molecule in the immune system, and that it is a 15.5-16 kDa protein that regulates the activities of white blood cells (leukocytes, often lymphocytes) that are responsible for immunity. IL-2 is understood to be part of the body's natural response to microbial infection and to mediate its effects by binding to IL-2 receptors, which are expressed by lymphocytes. Major sources of IL-2 are activated CD4+ T cells and activated CD8+ T cells.

IL-2 is thought to have essential roles in key functions of the immune system, tolerance and immunity, primarily via its direct effects on T cells. In the thymus, where T cells mature, it prevents autoimmune diseases by promoting the differentiation of certain immature T cells into regulatory T cells, which suppress other T cells that are otherwise primed to attack normal healthy cells in the body. IL-2 enhances activation-induced cell death (AICD). IL-2 also promotes the differentiation of T cells into effector T cells and into memory T cells when the initial T cell is also stimulated by an antigen, thus helping the body fight off infections. Together with other polarizing cytokines, IL-2 stimulates naive CD4+ T cell differentiation into Th1 and Th2 lymphocytes while it impedes differentiation into Th17 and follicular Th lymphocytes. Its expression and secretion is tightly regulated and functions as part of both transient positive and negative feedback loops in mounting and dampening immune responses. Through its role in the development of T cell immunologic memory, which depends upon the expansion of the number and function of antigen-selected T cell clones, it plays a role in enduring cell-mediated immunity.

In some aspects, Tregs are cultured in the presence of an interleukin (e.g., IL-2). The interleukin (e.g., IL-2) can be recombinant interleukin (e.g., IL-2). In some aspects, the concentration of the interleukin (e.g., IL-2) is at least 400 units/mL. In some aspects, the concentration of the interleukin (e.g., IL-2) is at least 500 units/mL. In some aspects, the concentration of the interleukin (e.g., IL-2) is at least 550 units/mL. In some aspects, the concentration of the interleukin (e.g., IL-2) is at least 600 units/mL.

In some aspects, the concentration of the interleukin (e.g., IL-2) is less than or equal to 1,000 units/mL. In some aspects, the concentration of the interleukin (e.g., IL-2) is less than or equal to 900 units/mL. In some aspects, the concentration of the interleukin (e.g., IL-2) is less than or equal to 800 units/mL.

In some aspects, the concentration of the interleukin (e.g., IL-2) is about 200 units/mL to about 2,500 units/mL. In some aspects, the concentration of the interleukin (e.g., IL-2) is about 500 units/mL to about 1,000 units/mL. In some aspects, the concentration of the interleukin (e.g., IL-2) is about 500 units/mL to about 900 units/mL. In some aspects, the concentration of the interleukin (e.g., IL-2) is about 500 units/mL to about 800 units/mL. In some aspects, the concentration of the interleukin (e.g., IL-2) is about 550 units/mL to about 1,000 units/mL. In some aspects, the concentration of the interleukin (e.g., IL-2) is about 550 units/mL to about 900 units/mL. In some aspects, the concentration of the interleukin (e.g., IL-2) is about 550 units/mL to about 800 units/mL. In some aspects, the concentration of the interleukin (e.g., IL-2) is about 600 units/mL to about 1,000 units/mL. In some aspects, the concentration of the interleukin (e.g., IL-2) is about 600 units/mL to about 900 units/mL. In some aspects, the concentration of the interleukin (e.g., IL-2) is about 600 units/mL to about 800 units/mL.

The concentration of the interleukin (e.g., IL-2) can be altered during the methods provided here. For example, the concentration of the interleukin (e.g., IL-2) can be reduced. In some aspects, the interleukin (e.g., IL-2) is present at a concentration of about 800 units/mL and then reduced to a concentration of about 300 units/mL. In some aspects, the interleukin (e.g., IL-2) is present at a concentration of about 800 units/mL for about 7 days (about 168 hours) and then at about 300 units/mL.

The term “IL-7” (also referred to herein as “IL7”) is a cytokine secreted by stromal cells in the bone marrow and thymus. It is also produced by keratinocytes, dendritic cells, hepatocytes, neurons, and epithelial cells, but is not produced by normal lymphocytes. IL-7 stimulates the differentiation of multipotent (pluripotent) hematopoietic stem cells into lymphoid progenitor cells (as opposed to myeloid progenitor cells where differentiation is stimulated by IL-3). IL-7 has been reported to stimulate proliferation of all cells in the lymphoid lineage (B cells, T cells and NK cells). It is believed to be important for proliferation during certain stages of B-cell maturation, T and NK cell survival, development and homeostasis. An example nucleotide sequence for a human IL7 gene is the NCBI Reference Sequence: AH006906.2. The amino acid sequence of a recombinant human IL-7 suitable for use in the methods provided herein is:

(SEQ ID NO: 5)
MDCDIEGKDGKQYESVLMVSIDQLLDSMKEIGSNCLNNEFNFFKRHICD
ANKEGMFLFRAARKLRQFLKMNSTGDFDLHLLKVSEGTTILLNCTGQVK
GRKPAALGEAQPTKSLEENKSLKEQKKLNDLCFLKRLLQEIKTCWNKIL
MGTKEH.

As utilized in the methods provided herein, a concentration of IL-7 can be from about 10 U/ml to about 7,000 U/ml. In some aspects, the concentration of IL-7 can be from about 5 ng/ml to about 3,500 ng/ml.

The term “IL-15” (also referred to herein as “IL15”) refers to the cytokine and T cell growth factor known as interleukin-15, and as utilized in the present invention, includes all forms of IL-15, including human and other mammalian forms, forms with conservative amino acid substitutions, glycoforms, biosimilars, and variants thereof. IL-15 is described, e.g., in Steel JC, Waldmann TA, Morris JC (January 2012) “Interleukin-15 biology and its therapeutic implications in cancer,” Trends in Pharmacological Sciences, 33 (1): 35-41 and Waldmann TA, Tagaya Y (1999) “The multifaceted regulation of interleukin-15 expression and the role of this cytokine in NK cell differentiation and host response to intracellular pathogens,” Annual Review of Immunology, 17: 19-49, the disclosures of which are incorporated herein by reference in their entireties. The term IL-15 also encompasses recombinant forms of IL-15. As used herein, the term IL-15 also encompasses pegylated forms of IL-15. The human IL15 gene is identified by NCBI Gene ID 3600. An example nucleotide sequence for a human IL15 gene is the NCBI Reference Sequence: NG_029605.2. The amino acid sequence of recombinant human IL-15 suitable for use in the methods provided herein is:

(SEQ ID NO: 6)
MNWVNVISDLKKIEDLIQSMHIDATLYTESDVHPSCKVTAMKCFLLELQ
VISLESGDASIHDTVENLIILANNSLSSNGNVTESGCKECEELEEKNIK
EFLQSFVHIVQMFINTS.

IL-15 can be utilized in the methods provided herein at a concentration of greater than 0.5 ng/ml. In some aspects, the concentration of IL-15 utilized is more than 1 ng/ml. In some aspects, the concentration of IL-15 utilized is more than 2 ng/ml. In some aspects, the concentration of IL-15 utilized is more than 10 ng/ml. In some aspects, the concentration of IL-15 utilized is more than 50 ng/ml. In some aspects, the concentration of IL-15 utilized is more than 75 ng/ml. In some aspects, the concentration of IL-15 utilized is more than 100 ng/ml. In some aspects, the concentration of IL-15 utilized is more than 150 ng/ml. In some aspects, the concentration of IL-15 utilized is more than 200 ng/ml. In some aspects, the concentration of IL-15 utilized is less than 10,000 ng/ml, optionally less than 9000, 8000, 7000, 6000, 5000, 4000, 3000, 2000, or 1000 ng/ml. In some aspects, the concentration of IL-15 utilized is about 300 ng/ml. In some aspects, the concentration of IL-15 utilized is about 1000 ng/ml. In some aspects, the concentration of IL-15 utilized is greater than 1000 ng/ml. In some aspects, the concentration of the IL-15 is greater than 100 ng/ml. In some aspects, the concentration of IL-15 is about 100 ng/ml to about 1000 ng/ml. In some aspects, the concentration of IL-15 is about 300 ng/ml.

IL-15 can be utilized in the methods provided at a concentration of greater than 1 U/ml. In some aspects, the concentration of IL-15 utilized is more than 2 U/ml. In some aspects, the concentration of IL-15 utilized is more than 4 U/ml. In some aspects, the concentration of IL-15 utilized is more than 20 U/ml. In some aspects, the concentration of IL-15 utilized is more than 200 U/ml. In some aspects, the concentration of IL-15 utilized is less than 20,000 U/ml, optionally less than 18,000, 16,000, 14,000, 12,000, 10,000, 8000, 6000, 4000, or 2000 ng/ml. In some aspects, the concentration of IL-15 utilized is about 600 U/ml. In some aspects, the concentration of IL-15 utilized is about 2000 U/ml. In some aspects, the concentration of IL-15 utilized is greater than 2000 U/ml. In some aspects, the concentration of the IL-15 is greater than 200 U/ml. In some aspects, the concentration of IL-15 is 200 U/ml to about 2000 U/ml. In some aspects, the concentration of IL-15 is about 600 U/ml.

In some aspects, Tregs are cultured in the presence of N-Acetyl-L-cysteine (NAC). In some aspects, the NAC is present at a concentration of about 5 mM in the culture. In some aspects, the NAC is present at a concentration of about 8.2 mg/ml in the culture.

In some aspects, Tregs are cultured at a concentration of about 0.125 million cells per ml. In some aspects, Tregs are cultured at a concentration of about 0.25 million cells per ml. In some aspects, Tregs are cultured at a concentration of about 0.5 million cells per ml.

In some aspects, Tregs are cultured at a concentration of about 0.125 million cells per ml to about 5 million cells per ml. In some aspects, Tregs are cultured at a concentration of about 0.25 million cells per ml to about 5 million cells per ml. In some aspects, Tregs are cultured at a concentration of about 0.5 million cells per ml to about 5 million cells per ml.

In some aspects, Tregs are cultured at a concentration of about 0.125 million cells per ml to about 3 million cells per ml. In some aspects, Tregs are cultured at a concentration of about 0.25 million cells per ml to about 3 million cells per ml. In some aspects, Tregs are cultured at a concentration of about 0.5 million cells per ml to about 3 million cells per ml.

In some aspects, Tregs are cultured at a concentration of about 0.125 million cells per ml to about 2.5 million cells per ml. In some aspects, Tregs are cultured at a concentration of about 0.25 million cells per ml to about 2.5 million cells per ml. In some aspects, Tregs are cultured at a concentration of about 0.5 million cells per ml to about 2.5 million cells per ml.

In some aspects, Tregs are cultured at a concentration of about 0.125 million cells per ml to about 2 million cells per ml. In some aspects, Tregs are cultured at a concentration of about 0.25 million cells per ml to about 2 million cells per ml. In some aspects, Tregs are cultured at a concentration of about 0.5 million cells per ml to about 2 million cells per ml.

In some aspects, Tregs are cultured at a concentration of about 0.125 million cells per ml to about 1.5 million cells per ml. In some aspects, Tregs are cultured at a concentration of about 0.25 million cells per ml to about 1.5 million cells per ml. In some aspects, Tregs are cultured at a concentration of about 0.5 million cells per ml to about 1.5 million cells per ml.

In some aspects, Tregs are cultured at a concentration of about 0.125 million cells per ml to about 1 million cells per ml. In some aspects, Tregs are cultured at a concentration of about 0.25 million cells per ml to about 1 million cells per ml. In some aspects, Tregs are cultured at a concentration of about 0.5 million cells per ml to about 1 million cells per ml.

In some aspects, Tregs are cultured at a concentration that does not exceed 5 million cells per ml. In some aspects, Tregs are cultured at a concentration that does not exceed 3 million cells per ml. In some aspects, Tregs are cultured at a concentration that does not exceed 2.5 million cells per ml. In some aspects, Tregs are cultured at a concentration that does not exceed 2 million cells per ml. In some aspects, Tregs are cultured at a concentration that does not exceed 1.5 million cells per ml. In some aspects, Tregs are cultured at a concentration that does not exceed 1 million cells per ml.

In some aspects, Tregs are cultured at a temperature suitable for the growth of T cells, for example, at least 25 degrees Celsius, at least 30 degrees Celsius, or about 37 degrees Celsius.

In some aspects, the methods comprise the use of rapamycin, which can limit the growth of effector T cells (Teffs). Rapamycin can be used, e.g., in the first week of culture. In some aspects, rapamycin can be used in the first two weeks of culture. In some aspects, the methods do not comprise the use of rapamycin. In some aspects, the methods do not comprise the use of rapamycin prior to genetic engineering. In some aspects, the methods do not comprise the use of rapamycin in the first two weeks of culture. In some aspects, the methods do not comprise the use of rapamycin. In the first week of culture.

In some aspects, the methods provided herein comprise the use of supermagnetic beads. In some aspects, the methods do not comprise the use of supermagnetic beads. In some aspects, the methods do not comprise the use of beads.

In some aspects, the methods provided herein comprise the use of an artificial antigen presenting cell. In some aspects, the methods provided herein do not comprise the use of an artificial antigen presenting cell.

In some aspects, a method of expanding Tregs provided herein comprises: (a) culturing Tregs in the presence of a stimulating agent (e.g., a tetrameric antibody complex that specifically binds to CD3, CD28, and CD2, optionally at a concentration of 10 μl/ml) for about 3 days (about 72 hours) (e.g., Day 0 to Day 3); and (b) removing the stimulating agent (e.g., a tetrameric antibody complex that specifically binds to CD3, CD28, and CD2), optionally by washing; replating the cells, optionally at 0.5 million cells per ml; and culturing the cells in the absence of a stimulating agent for about 3 days (about 72 hours) (e.g., Day 3 to Day 6), wherein the method results in at least 40-fold, at least 50-fold at least 60-fold, at least 70-fold, or about 80-fold expansion of the Tregs.

In some aspects, a method of expanding Tregs provided herein comprises:
(a) culturing Tregs in the presence of a stimulating agent (e.g., a tetrameric antibody complex that specifically binds to CD3, CD28, and CD2, optionally at a concentration of 10 μl/ml) for about 3 days (about 72 hours) (e.g., Day 0 to Day 3);
(b) removing the stimulating agent (e.g., a tetrameric antibody complex that specifically binds to CD3, CD28, and CD2), optionally by washing; replating the cells, optionally at 0.5 million cells per ml; and culturing the cells in the absence of a stimulating agent for about 3 days (about 72 hours) (e.g., Day 3 to Day 6);
(c) culturing Tregs in the presence of a stimulating agent (e.g., a tetrameric antibody complex that specifically binds to CD3, CD28, and CD2, optionally at a concentration of 10 μl/ml) for about 2 days (about 48 hours) (e.g., Day 6 to Day 8); adjusting the concentration of cells to 0.5 million cells per ml;
(d) removing the stimulating agent (e.g., a tetrameric antibody complex that specifically binds to CD3, CD28, and CD2), optionally by washing; genetically engineering the Tregs; and culturing the culturing the cells in the absence of a stimulating agent for about 3 days (about 72 hours) (e.g., Day 8 to Day 11);
(e) culturing Tregs in the presence of a stimulating agent (e.g., a tetrameric antibody complex that specifically binds to CD3, CD28, and CD2, optionally at a concentration of 10 μl/ml) for about 2 days (about 48 hours) (e.g., Day 11 to Day 13);
(f) removing the stimulating agent (e.g., a tetrameric antibody complex that specifically binds to CD3, CD28, and CD2), optionally by washing; replating the cells, optionally at 0.5 million cells per ml; and culturing the cells in the absence of a stimulating agent for about 2 days (about 48 hours) (e.g., Day 13 to Day 15); and
(g) harvesting the cells.

Such a method comprising steps (a)-(g) can comprise culturing the Tregs in the presence of IL-2 (optionally at a concentration of 400 IU/ml) and/or NAC (optionally at a concentration of 8.2 mg/ml). In some aspects, the concentration of IL-2 is adjusted to 400 IU/ml on Days 2-9, 11, 13, and 14.

Such a method comprising steps (a)-(g) can comprise splitting the cells multiple times, for example on Days 4, 5, 7, 11, 13, and 14.

In some aspects, the Tregs have expanded by at least 60-fold or by at least 70-fold by the end of step (b), e.g., by Day 6. 80-fold by the end of step (b), e.g., by Day 6.

In some aspects, the Tregs have expanded by about 80-fold by the end of step (b), e.g., by Day 6.

In some aspects, step (c) further comprises transferring the Tregs to T25 flasks.

Using the methods of expansion provided herein, the number of Tregs in a Treg population can be expanded by at least 500-fold, by at least 1000-fold, by at least 1500-fold, be at least 2000-fold, by at least 2500-fold, by at least 3000-fold, by at least 3500-fold, or by at least 4000 fold.

In some aspects, the number of Tregs is expanded by at least 500-fold within 10 days.

In some aspects, the number of Tregs is expanded by at least 1000-fold within 20 days. In some aspects, the number of Tregs is expanded by at least 1000-fold within 19 days. In some aspects, the number of Tregs is expanded by at least 1000-fold within 18 days. In some aspects, the number of Tregs is expanded by at least 1000-fold within 17 days. In some aspects, the number of Tregs is expanded by at least 1000-fold within 16 days. In some aspects, the number of Tregs is expanded by at least 1000-fold within 15 days. In some aspects, the number of Tregs is expanded by at least 1000-fold within 14 days. In some aspects, the number of Tregs is expanded by at least 1000-fold within 13 days. In some aspects, the number of Tregs is expanded by at least 1000-fold within 12 days. In some aspects, the number of Tregs is expanded by at least 1000-fold within 11 days.

In some aspects, the number of Tregs is expanded by at least 2500-fold within 20 days. In some aspects, the number of Tregs is expanded by at least 3000-fold within 20 days. In some aspects, the number of Tregs is expanded by at least 3500-fold within 20 days. In some aspects, the number of Tregs is expanded by at least 4000-fold within 20 days.

The Tregs produced according to the methods provided herein have improved properties, e.g., because the methods provided herein prevent overstimulation of the population of Tregs and/or prevent activation-induced cell death. For example, in some aspects, Tregs produced according to the methods provided herein have smaller surface area, e.g., as compared to Tregs that are cultured in the presence of a stimulating agent for 6 days (about 144 hours). In some aspects, Tregs produced according to the methods provided herein have a surface area that is less than 0.9, less than 0.85, less than 0.8, or less than 0.75 times the surface area of Tregs that are cultured in the presence of a stimulating agent for 6 days (about 144 hours). In some aspects, Tregs produced according to the methods provided herein have an increased proportion of Helios+Foxp3+ Tregs, e.g., as compared to Tregs that are cultured in the presence of a stimulating agent for 6 days (about 144 hours). In some aspects, Tregs produced according to the methods provided herein have an increased fold expansion, e.g., as compared to Tregs that are cultured in the presence of a stimulating agent for 6 days (about 144 hours). In some aspects, Tregs produced according to the methods provided herein have a fold expansion that is at least 1.5 times or at least 2 times the fold expansion of Tregs cultured in the presence of a stimulating agent for 6 days (about 144 hours). In some aspects, Tregs produced according to the methods provided herein have an increased ability to suppress proliferation of effector T cells (Teffs), e.g., as compared to Tregs that are cultured in the presence of a stimulating agent for 6 days (about 144 hours). In some aspects, Tregs produced according to the methods provided herein have an at least 6-fold, at least 7-fold, at least 8-fold, at least 9-fold, or at least 10-fold increased ability to suppress proliferation of Teffs, e.g., as compared to Tregs that are cultured in the presence of a stimulating agent for 6 days (about 144 hours).

In some aspects, Tregs expanded according to the methods provided herein maintain at least 60%, at least 70%, at least 80%, or at least 90% of the Helios expression as compared to the population of Tregs prior to the expansion.

In some aspects, Tregs produced according to the methods provided herein continue to expand after genetic engineering. In some aspects, Tregs produced according to the methods provided herein expand at least 1.5-fold after genetic engineering (e.g., within 3 days (about 72 hours) or within 4 days (about 96 hours) after genetic engineering). In some aspects, Tregs produced according to the methods provided herein expand at least 2-fold after genetic engineering (e.g., within 3 days (about 72 hours) or within 4 days (about 96 hours) after genetic engineering). In some aspects, Tregs produced according to the methods provided herein expand at least 3-fold after genetic engineering (e.g., within 3 days (about 72 hours) or within 4 days (about 96 hours) after genetic engineering).

In some aspects, the methods provided herein comprise harvesting an expanded (and optionally genetically engineered) population of Tregs. In some aspects, the harvesting immediately follows a stimulating step. In some aspects, the harvesting immediately follows a resting step.

In some aspects, a stimulating step occurs after genetic engineering and prior to harvesting. In some aspects, a resting step occurs after genetic engineering and prior to harvesting. In some aspects, both a stimulating step and a resting step occur after genetic engineering and prior to harvesting.

In some aspects, the methods provided herein comprise cryopreserving an expanded (and optionally genetically engineered) population of Tregs. In some aspects, the cryopreserving immediately follows a stimulating step. In some aspects, the cryopreserving immediately follows a resting step.

### Genetic Engineering of Tregs

In some aspects of the methods provided herein, Tregs are genetically engineered. Genetic engineering can comprise, e.g., introducing a nucleic acid or a gene-regulating system into a Treg or population of Tregs. The nucleic acid or gene-regulating system can be introduced using methods known in the art, including for example, by electroporation and/or or Ribonucleoprotein (RNP)-mediated methods.

A nucleic acid that can be introduced into Tregs via genetic engineering can be a viral nucleic acid or a non-viral nucleic acid.

A nucleic acid that is introduced into a Treg or population or Tregs can be a nucleic acid that encodes a protein. The protein can be a protein that is heterologous to the Treg(s). For example, the protein can be an engineered antigen receptor such as a chimeric antigen receptor (CAR) or an engineered TCR.

In some aspects, the engineered antigen receptor is a CAR comprising an extracellular antigen binding domain fused via hinge and transmembrane domains to a cytoplasmic domain comprising a signaling domain. In some aspects, the extracellular domain of a CAR comprises an antigen binding fragment derived from an antibody. Antigen binding domains that are useful in the present disclosure include, for example, scFvs. In some aspects, the intracellular signaling domain of a CAR can be derived from the TCR complex zeta chain (such as CD3 signaling domains), FcyRIII, FccRI, or the T-lymphocyte activation domain. In some aspects, the intracellular signaling domain of a CAR further comprises a costimulatory domain, for example a 4-1BB, CD28, CD40, MyD88, or CD70 domain. In some aspects, the intracellular signaling domain of a CAR comprises two costimulatory domains, for example any two of 4-1BB, CD28, CD40, MyD88, or CD70 domains. Exemplary CAR structures and intracellular signaling domains are known in the art (See e.g., WO 2009/091826; US 20130287748; WO 2015/142675; WO 2014/055657; and WO 2015/090229, incorporated herein by reference).

CARs specific for antigens relevant for autoimmune diseases (e.g., GVHD, colitis, and multiple sclerosis) are discussed, for example, in Zhang et al., Frontiers in Immunology 9:1-8 (2018); Int'l Publ. No. W02017218850A1; and MacDonald et al., JCI 2016;126(4):1413-1424, each of which is incorporated by reference herein in its entirety.

In some aspects, the engineered antigen receptor is an engineered TCR. Engineered TCRs comprise TCRα and/or TCRβ chains that have been isolated and cloned from T cell populations recognizing a particular target antigen. Engineered TCRs recognize antigen through the same mechanisms as their endogenous counterparts (e.g., by recognition of their cognate antigen presented in the context of major histocompatibility complex (MHC) proteins expressed on the surface of a target cell). This antigen engagement stimulates endogenous signal transduction pathways leading to activation and proliferation of the TCR-engineered cells.

Herein, the term “gene-regulating system” refers to a protein, nucleic acid, or combination thereof that is capable of modifying an endogenous target DNA sequence when introduced into a cell, thereby regulating the expression or function of the encoded gene product. Numerous gene editing systems suitable for use in the methods of the present disclosure are known in the art including, but not limited to, shRNAs, siRNAs, zinc-finger nuclease systems, TALEN systems, and CRISPR/Cas systems. Gene-regulating systems useful in the methods herein are provided, for example, in WO 2020/160489, which is herein incorporated by reference in its entirely.

As used herein, “regulate,” when used in reference to the effect of a gene-regulating system on an endogenous target gene encompasses any change in the sequence of the endogenous target gene, any change in the epigenetic state of the endogenous target gene, and/or any change in the expression or function of the protein encoded by the endogenous target gene.

In some aspects, the gene-regulating system can mediate a change in the sequence of an endogenous target gene, for example, by introducing one or more mutations into the endogenous target sequence, such as by insertion or deletion of one or more nucleic acids in the endogenous target sequence. Exemplary mechanisms that can mediate alterations of the endogenous target sequence include, but are not limited to, non-homologous end joining (NHEJ) (e.g., classical or alternative), microhomology-mediated end joining (MMEJ), homology-directed repair (e.g., endogenous donor template mediated), SDSA (synthesis dependent strand annealing), single strand annealing or single strand invasion.

In some aspects, the gene-regulating system can mediate a change in the epigenetic state of an endogenous target sequence. For example, in some aspects, the gene-regulating system can mediate covalent modifications of an endogenous target gene DNA (e.g., cytosine methylation and hydroxymethylation) or of associated histone proteins (e.g. lysine acetylation, lysine and arginine methylation, serine and threonine phosphorylation, and lysine ubiquitination and sumoylation).

In some aspects, the gene-regulating system can mediate a change in the expression of a protein encoded by an endogenous target gene. In such aspects, the gene-regulating system can regulate the expression of the encoded protein by modifications of the endogenous target DNA sequence, or by acting on the mRNA product encoded by the DNA sequence. In some aspects, the gene-regulating system can result in the expression of a modified endogenous protein. In such aspects, the modifications to the endogenous DNA sequence mediated by the gene-regulating system result in the expression of an endogenous protein demonstrating a reduced function as compared to the corresponding endogenous protein in a non-genetically engineered Treg. In such aspects, the expression level of the modified endogenous protein can be increased, decreased can may be the same, or substantially similar to, the expression level of the corresponding endogenous protein in an non-genetically engineered Treg.

Gene-regulating systems that can be introduced into Tregs via genetic engineering can comprise (i) a nucleic acid molecule; (ii) an enzymatic protein; or (iii) a nucleic acid molecule and an enzymatic protein. Such a gene-regulating system can comprise a nucleic acid molecule selected from an siRNA, an shRNA, a microRNA (miR), an antagomiR, or an antisense RNA. Such a gene-regulating system can comprise an enzymatic protein that has been engineered to specifically bind to a target sequence in one or more genes in the Tregs. The enzymatic protein can be, for example, a Transcription activator-like effector nuclease (TALEN), a zinc-finger nuclease, or a meganuclease. Such a gene-regulating system can comprise a nucleic acid molecule and an enzymatic protein, wherein the nucleic acid molecule is a guide RNA (gRNA) molecule and the enzymatic protein is a Cas protein or Cas ortholog. The Cas protein can be a Cas9 protein.

### Nucleic Acid-Based Gene-Regulating Systems

As used herein, a nucleic acid-based gene-regulating system is a system comprising one or more nucleic acid molecules that is capable of regulating the expression of an endogenous target gene without the requirement for an exogenous protein. In some aspects, the nucleic acid-based gene-regulating system comprises an RNA interference molecule or antisense RNA molecule that is complementary to a target nucleic acid sequence.

An “antisense RNA molecule” refers to an RNA molecule, regardless of length, that is complementary to an mRNA transcript. Antisense RNA molecules refer to single stranded RNA molecules that can be introduced to a cell, tissue, or subject and result in decreased expression of an endogenous target gene product through mechanisms that do not rely on endogenous gene silencing pathways, but rather rely on RNaseH-mediated degradation of the target mRNA transcript. In some aspects, an antisense nucleic acid comprises a modified backbone, for example, phosphorothioate, phosphorodithioate, or others known in the art, or may comprise non-natural internucleoside linkages. In some aspects, an antisense nucleic acid can comprise locked nucleic acids (LNA).

“RNA interference molecule” as used herein refers to an RNA polynucleotide that mediates the decreased the expression of an endogenous target gene product by degradation of a target mRNA through endogenous gene silencing pathways (e.g., Dicer and RNA-induced silencing complex (RISC)). Exemplary RNA interference agents include micro RNAs (also referred to herein as “miRNAs”), short hair-pin RNAs (shRNAs), small interfering RNAs (siRNAs), RNA aptamers, and morpholinos.

In some aspects, the nucleic acid-based gene-regulating system comprises one or more miRNAs. miRNAs refers to naturally occurring, small non-coding RNA molecules of about 21-25 nucleotides in length. miRNAs are at least partially complementary to one or more target mRNA molecules. miRNAs can downregulate (e.g., decrease) expression of an endogenous target gene product through translational repression, cleavage of the mRNA, and/or deadenylation.

In some aspects, the nucleic acid-based gene-regulating system comprises one or more shRNAs. shRNAs are single stranded RNA molecules of about 50-70 nucleotides in length that form stem-loop structures and result in degradation of complementary mRNA sequences. shRNAs can be cloned in plasmids or in non-replicating recombinant viral vectors to be introduced intracellularly and result in the integration of the shRNA-encoding sequence into the genome. As such, an shRNA can provide stable and consistent repression of endogenous target gene translation and expression.

In some aspects, nucleic acid-based gene-regulating system comprises one or more siRNAs. siRNAs refer to double stranded RNA molecules typically about 21-23 nucleotides in length. The siRNA associates with a multi protein complex called the RNA-induced silencing complex (RISC), during which the “passenger” sense strand is enzymatically cleaved. The antisense “guide” strand contained in the activated RISC then guides the RISC to the corresponding mRNA because of sequence homology and the same nuclease cuts the target mRNA, resulting in specific gene silencing. Optimally, an siRNA is 18, 19, 20, 21, 22, 23 or 24 nucleotides in length and has a 2 base overhang at its 3′ end. siRNAs can be introduced to an individual cell and/or culture system and result in the degradation of target mRNA sequences. siRNAs and shRNAs are further described in Fire et al., Nature, 391:19, 1998 and U.S. Pat. Nos. 7,732,417; 8,202,846; and 8,383,599.

In some aspects, the nucleic acid-based gene-regulating system comprises one or more morpholinos. “Morpholino” as used herein refers to a modified nucleic acid oligomer wherein standard nucleic acid bases are bound to morpholine rings and are linked through phosphorodiamidate linkages. Similar to siRNA and shRNA, morpholinos bind to complementary mRNA sequences. However, morpholinos function through steric-inhibition of mRNA translation and alteration of mRNA splicing rather than targeting complementary mRNA sequences for degradation.

In some aspects, the nucleic acid-based gene-regulating system comprises a nucleic acid molecule (e.g., an siRNA, an shRNA, an RNA aptamer, or a morpholino) that binds to a target RNA sequence that is at least 90% identical to an RNA encoded by a DNA sequence of a target gene selected from those listed in Table 1. In some aspects, the nucleic acid-based gene-regulating system comprises a nucleic acid molecule (e.g., an siRNA, an shRNA, an RNA aptamer, or a morpholino) that bind to a target RNA sequence that is at least 95%, 96%, 97%, 98%, or 99% identical to an RNA encoded by a DNA sequence of a target gene selected from those listed in Table 1. In some aspects, the nucleic acid-based gene-regulating system comprises a nucleic acid molecule (e.g., an siRNA, an shRNA, an RNA aptamer, or a morpholino) bind to a target RNA sequence that is 100% identical to an RNA encoded by a DNA sequence of a target gene selected from those listed in Table 1.

TABLE 1
Exemplary Endogenous Genes

| Gene | | Human UniProt | Murine UniProt |
|---|---|---|---|
| Symbol | Gene Name | Ref. | Ref. |
| PRDM1 | PR domain zinc finger | O75626 | Q60636 |
| | protein 1 | | |
| TNFRSF4 | Tumor necrosis factor | P43489 | P47741 |
| | receptor superfamily, | | |
| | member 4 | | |
| REEP3 | Receptor Accessory | Q6NUK4 | Q99KK1 |
| | Protein 3 | | |
| MRPL32 | 39S ribosomal protein L32, | Q9BYC8 | Q9DCI9 |
| | mitochondrial | | |
| FSCN3 | Fascin-3 | Q9NQT6 | Q9QXW4 |
| KLC3 | Kinesin light chain 3 | Q6P597 | Q91W40 |
| C4BPA | Complement Component 4 | P04003 | P08607 |
| | Binding Protein Alpha | | |
| LZTS1 | Leucine zipper putative | Q9Y250 | P60853 |
| | tumor suppressor 1 | | |
| CDK16 | Cyclin Dependent Kinase | Q00536 | Q04735 |
| | 16 | | |
| ADNP | Activity Dependent | Q9H2P0 | Q9Z103 |
| | Neuroprotector Homeobox | | |

In some aspects, the nucleic acid-based gene-regulating system comprises an siRNA molecule or an shRNA molecule selected from those known in the art, such as the siRNA and shRNA constructs available from commercial suppliers such as Sigma Aldrich, Dharmacon, ThermoFisher, and the like.

In some aspects, the gene-regulating system comprises two or more nucleic acid molecules (e.g., two or more siRNAs, two or more shRNAs, two or more RNA aptamers, or two or more morpholinos), wherein at least one of the nucleic acid molecules binds to a target RNA sequence that is at least 90% identical to an RNA sequence encoded by a DNA sequence of a target gene selected from Table 1. In some aspects, the gene-regulating system comprises two or more nucleic acid molecules (e.g., two or more siRNAs, two or more shRNAs, two or more RNA aptamers, or two or more morpholinos), wherein at least one of the nucleic acid molecules binds to a target RNA sequence that is at least 95%, 96%, 97%, 98%, or 99% identical to an RNA sequence encoded by a DNA sequence of a target gene selected from Table 1. In some aspects, the gene-regulating system comprises two or more nucleic acid molecules (e.g., two or more siRNAs, two or more shRNAs, two or more RNA aptamers, or two or more morpholinos), wherein at least one of the nucleic acid molecules binds to a target RNA sequence that is 100% identical to an RNA sequence encoded by a DNA sequence of a target gene selected from Table 1.

### Protein-Based Gene-Regulating Systems

In some aspects, a protein-based gene-regulating system is a system comprising

one or more proteins capable of regulating the expression of an endogenous target gene in a sequence specific manner without the requirement for a nucleic acid guide molecule. In some aspects, the protein-based gene-regulating system comprises a protein comprising one or more zinc-finger binding domains and an enzymatic domain. In some aspects, the protein-based gene-regulating system comprises a protein comprising a Transcription activator-like effector nuclease (TALEN) domain and an enzymatic domain. Such aspects are referred to herein as “TALENs.”

### 1. Zinc Finger Systems

Zinc finger-based systems comprise a fusion protein comprising two protein domains: a zinc finger DNA binding domain and an enzymatic domain. A “zinc finger DNA binding domain”, “zinc finger protein”, or “ZFP” is a protein, or a domain within a larger protein, that binds DNA in a sequence-specific manner through one or more zinc fingers, which are regions of amino acid sequence within the binding domain whose structure is stabilized through coordination of a zinc ion. The zinc finger domain, by binding to a target DNA sequence, directs the activity of the enzymatic domain to the vicinity of the sequence and, hence, induces modification of the endogenous target gene in the vicinity of the target sequence. A zinc finger domain can be engineered to bind to virtually any desired sequence. Accordingly, after identifying a target genetic locus containing a target DNA sequence at which cleavage or recombination is desired (e.g., a target locus in a target gene referenced in Table 1), one or more zinc finger binding domains can be engineered to bind to one or more target DNA sequences in the target genetic locus. Expression of a fusion protein comprising a zinc finger binding domain and an enzymatic domain in a cell, effects modification in the target genetic locus.

In some aspects, a zinc finger binding domain comprises one or more zinc fingers. Miller et al. (1985) EMBO J. 4:1609-1614; Rhodes (1993) Scientific American Febuary:56-65; U.S. Pat. No. 6,453,242. Typically, a single zinc finger domain is about 30 amino acids in length. An individual zinc finger binds to a three-nucleotide (i.e., triplet) sequence (or a four-nucleotide sequence which can overlap, by one nucleotide, with the four-nucleotide binding site of an adjacent zinc finger). Therefore the length of a sequence to which a zinc finger binding domain is engineered to bind (e.g., a target sequence) will determine the number of zinc fingers in an engineered zinc finger binding domain. For example, for ZFPs in which the finger motifs do not bind to overlapping subsites, a six-nucleotide target sequence is bound by a two-finger binding domain; a nine-nucleotide target sequence is bound by a three-finger binding domain, etc. Binding sites for individual zinc fingers (i.e., subsites) in a target site need not be contiguous, but can be separated by one or several nucleotides, depending on the length and nature of the amino acids sequences between the zinc fingers (i.e., the inter-finger linkers) in a multi-finger binding domain. In some aspects, the DNA-binding domains of individual ZFNs comprise between three and six individual zinc finger repeats and can each recognize between 9 and 18 basepairs.

Zinc finger binding domains can be engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. An engineered zinc finger binding domain can have a novel binding specificity, compared to a naturally-occurring zinc finger protein. Engineering methods include, but are not limited to, rational design and various types of selection.

Selection of a target DNA sequence for binding by a zinc finger domain can be accomplished, for example, according to the methods disclosed in U.S. Pat. No. 6,453,242. It will be clear to those skilled in the art that simple visual inspection of a nucleotide sequence can also be used for selection of a target DNA sequence. Accordingly, any means for target DNA sequence selection can be used in the methods described herein. A target site generally has a length of at least 9 nucleotides and, accordingly, is bound by a zinc finger binding domain comprising at least three zinc fingers. However binding of, for example, a 4-finger binding domain to a 12-nucleotide target site, a 5-finger binding domain to a 15-nucleotide target site or a 6-finger binding domain to an 18-nucleotide target site, is also possible. As will be apparent, binding of larger binding domains (e.g., 7-, 8-, 9-finger and more) to longer target sites is also possible.

In some aspects, the zinc finger binding domains bind to a target DNA sequence that is at least 90% identical to a target DNA sequence of a target gene selected from those listed in Table 1. In some aspects, the zinc finger binding domains bind to a target DNA sequence that is at least 95%, 96%, 97%, 98%, or 99% identical to a target DNA sequence of a target gene selected from those listed in Table 1. In some aspects, the zinc finger binding domains bind to a target DNA sequence that is 100% identical to a target DNA sequence of a target gene selected from those listed in Table 1. In some aspects, the zinc finger system is selected from those known in the art, such as those available from commercial suppliers such as Sigma Aldrich.

In some aspects, the gene-regulating system comprises two or more ZFP-fusion proteins each comprising a zinc finger binding domain, wherein at least one of the zinc finger binding domains binds to a target DNA sequence that is at least 90% identical to a target DNA sequence of a target gene selected from Table 1. In some aspects, the gene-regulating system comprises two or more ZFP-fusion proteins each comprising a zinc finger binding domain, wherein at least one of the zinc finger binding domains binds to a target DNA sequence that is at least 95%, 96%, 97%, 98%, or 99% identical to a target DNA sequence of a target gene selected from Table 1. In some aspects, the gene-regulating system comprises two or more ZFP-fusion proteins each comprising a zinc finger binding domain, wherein at least one of the zinc finger binding domains binds to a target DNA sequence that is 100% identical to a target DNA sequence of a target gene selected from Table 1.

The enzymatic domain portion of the zinc finger fusion proteins can be obtained from any endo- or exonuclease. Exemplary endonucleases from which an enzymatic domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., 51 Nuclease; mung bean nuclease; pancreatic DNasel; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains.

Exemplary restriction endonucleases (restriction enzymes) suitable for use as an enzymatic domain of the ZFPs described herein are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in some aspects, fusion proteins comprise the enzymatic domain from at least one Type IIS restriction enzyme and one or more zinc finger binding domains.

An exemplary Type IIS restriction enzyme, whose cleavage domain is separable from the binding domain, is FokI. This particular enzyme is active as a dimer. Bitinaite et al. (1998) Proc. Natl. Acad. Sci. USA 95: 10,570-10,575. Thus, for targeted double-stranded DNA cleavage using zinc finger-FokI fusions, two fusion proteins, each comprising a FokI enzymatic domain, can be used to reconstitute a catalytically active cleavage domain. Alternatively, a single polypeptide molecule containing a zinc finger binding domain and two FokI enzymatic domains can also be used. Exemplary ZFPs comprising FokI enzymatic domains are described in U.S. Pat. No. 9,782,437.

### 2. TALEN Systems

TALEN-based systems comprise a protein comprising a TAL effector DNA binding domain and an enzymatic domain. They are made by fusing a TAL effector DNA-binding domain to a DNA cleavage domain (a nuclease which cuts DNA strands). The FokI restriction enzyme described above is an exemplary enzymatic domain suitable for use in TALEN-based gene-regulating systems.

TAL effectors are proteins that are secreted by Xanthomonas bacteria via their type III secretion system when they infect plants. The DNA binding domain contains a repeated, highly conserved, 33-34 amino acid sequence with divergent 12th and 13th amino acids. These two positions, referred to as the Repeat Variable Diresidue (RVD), are highly variable and strongly correlated with specific nucleotide recognition. Therefore, the TAL effector domains can be engineered to bind specific target DNA sequences by selecting a combination of repeat segments containing the appropriate RVDs. The nucleic acid specificity for RVD combinations is as follows: HD targets cytosine, NI targets adenenine, NG targets thymine, and NN targets guanine (though, in some aspects, NN can also bind adenenine with lower specificity).

In some aspects, the TAL effector domains bind to a target DNA sequence that is at least 90% identical to a target DNA sequence of a target gene selected from those listed in Table 1. In some aspects, the TAL effector domains bind to a target DNA sequence that is at least 95%, 96%, 97%, 98%, or 99% identical to a target DNA sequence of a target gene selected those listed in Table 1. In some aspects, the TAL effector domains bind to a target DNA sequence that is 100% identical to a target DNA sequence of a target gene selected from those listed in Table 1.

In some aspects, the gene-regulating system comprises two or more TAL effector-fusion proteins each comprising a TAL effector domain, wherein at least one of the TAL effector domains binds to a target DNA sequence that is at least 90% identical to a target DNA sequence of a target gene selected from Table 1. In some aspects, the gene-regulating system comprises two or more TAL effector-fusion proteins each comprising a TAL effector domain, wherein at least one of the TAL effector domains binds to a target DNA sequence that is at least 95%, 96%, 97%, 98%, or 99% identical to a target DNA sequence of a target gene selected from Table 1. In some aspects, the gene-regulating system comprises two or more TAL effector-fusion proteins each comprising a TAL effector domain, wherein at least one of the TAL effector domains binds to a target DNA sequence that is 100% identical to a target DNA sequence of a target gene selected from Table 1.

Methods and compositions for assembling the TAL-effector repeats are known in the art. See e.g., Cermak et al, Nucleic Acids Research, 39:12, 2011, e82. Plasmids for constructions of the TAL-effector repeats are commercially available from Addgene

### Combination Nucleic Acid/Protein-Based Gene-Regulating Systems

Combination gene-regulating systems comprise a site-directed modifying polypeptide and a nucleic acid guide molecule. Herein, a “site-directed modifying polypeptide” refers to a polypeptide that binds to a nucleic acid guide molecule, is targeted to a target nucleic acid sequence, (for example, an endogenous target DNA or RNA sequence) by the nucleic acid guide molecule to which it is bound, and modifies the target nucleic acid sequence (e.g., cleavage, mutation, or methylation of a target nucleic acid sequence).

A site-directed modifying polypeptide comprises two portions, a portion that binds the nucleic acid guide and an activity portion. In some aspects, a site-directed modifying polypeptide comprises an activity portion that exhibits site-directed enzymatic activity (e.g., DNA methylation, DNA or RNA cleavage, histone acetylation, histone methylation, etc.), wherein the site of enzymatic activity is determined by the guide nucleic acid. In some cases, a site-directed modifying polypeptide comprises an activity portion that has enzymatic activity that modifies the endogenous target nucleic acid sequence (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity). In other cases, a site-directed modifying polypeptide comprises an activity portion that has enzymatic activity that modifies a polypeptide (e.g., a histone) associated with the endogenous target nucleic acid sequence (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity). In some aspects, a site-directed modifying polypeptide comprises an activity portion that modulates transcription of a target DNA sequence (e.g., to increase or decrease transcription). In some aspects, a site-directed modifying polypeptide comprises an activity portion that modulates expression or translation of a target RNA sequence (e.g., to increase or decrease transcription).

The nucleic acid guide comprises two portions: a first portion that is complementary to, and capable of binding with, an endogenous target nucleic sequence (referred to herein as a “nucleic acid-binding segment”), and a second portion that is capable of interacting with the site-directed modifying polypeptide (referred to herein as a “protein-binding segment”). In some aspects, the nucleic acid-binding segment and protein-binding segment of a nucleic acid guide are comprised within a single polynucleotide molecule. In some aspects, the nucleic acid-binding segment and protein-binding segment of a nucleic acid guide are each comprised within separate polynucleotide molecules, such that the nucleic acid guide comprises two polynucleotide molecules that associate with each other to form the functional guide.

The nucleic acid guide mediates the target specificity of the combined

protein/nucleic acid gene-regulating systems by specifically hybridizing with a target nucleic acid sequence. In some aspects, the target nucleic acid sequence is an RNA sequence, such as an RNA sequence comprised within an mRNA transcript of a target gene. In some aspects, the target nucleic acid sequence is a DNA sequence comprised within the DNA sequence of a target gene. Reference herein to a target gene encompasses the full-length DNA sequence for that particular gene which comprises a plurality of target genetic loci (i.e., portions of a particular target gene sequence (e.g., an exon or an intron)). Within each target genetic loci are shorter stretches of DNA sequences referred to herein as “target DNA sequences” that can be modified by the gene-regulating systems described herein. Further, each target genetic loci comprises a “target modification site,” which refers to the precise location of the modification induced by the gene-regulating system (e.g., the location of an insertion, a deletion, or mutation, the location of a DNA break, or the location of an epigenetic modification).

The gene-regulating systems described herein may comprise a single nucleic acid guide, or may comprise a plurality of nucleic acid guides (e.g., 2, 3, 4, 5, 6, 7, 8, 9, 10, or more nucleic acid guides).

In some aspects, the combined protein/nucleic acid gene-regulating systems comprise site-directed modifying polypeptides derived from Argonaute (Ago) proteins (e.g., T. thermophiles Ago or TtAgo). In some aspects, the site-directed modifying polypeptide is a T. thermophiles Ago DNA endonuclease and the nucleic acid guide is a guide DNA (gDNA) (See, Swarts et al., Nature 507 (2014), 258-261). In some aspects, the present disclosure provides a polynucleotide encoding a gDNA. In some aspects, a gDNA-encoding nucleic acid is comprised in an expression vector, e.g., a recombinant expression vector. In some aspects, the present disclosure provides a polynucleotide encoding a TtAgo site-directed modifying polypeptide or variant thereof. In some aspects, the polynucleotide encoding a TtAgo site-directed modifying polypeptide is comprised in an expression vector, e.g., a recombinant expression vector.

In some aspects, the gene editing systems described herein are CRISPR (Clustered Regularly Interspaced Short Palindromic Repeats)/Cas (CRISPR Associated) nuclease systems. In some aspects, the CRISPR/Cas system is a Class 2 system. Class 2 CRISPR/Cas systems are divided into three types: Type II, Type V, and Type VI systems. In some aspects, the CRISPR/Cas system is a Class 2 Type II system, utilizing the Cas9 protein. In some aspects, the site-directed modifying polypeptide is a Cas9 DNA endonuclease (or variant thereof) and the nucleic acid guide molecule is a guide RNA (gRNA). In some aspects, the CRISPR/Cas system is a Class 2 Type V system, utilizing the Cas12 proteins (e.g., Cas12a (also known as Cpf1), Cas12b (also known as C2c1), Cas12c (also known as C2c3), Cas12d (also known as CasY), and Cas12e (also known as CasX)). In some aspects, the site-directed modifying polypeptide is a Cas12 DNA endonuclease (or variant thereof) and the nucleic acid guide molecule is a gRNA. In some aspects, the CRISPR/Cas system is a Class 2 and Type VI system, utilizing the Cas13 proteins (e.g., Cas13a (also known as C2c2), Cas13b, and Cas13c). (See, Pyzocha et al., ACS Chemical Biology, 13(2), 347-356). In some aspects, the site-directed modifying polypeptide is a Cas13 RNA riboendonuclease and the nucleic acid guide molecule is a gRNA.

A Cas polypeptide refers to a polypeptide that can interact with a gRNA molecule and, in concert with the gRNA molecule, home or localize to a target DNA or target RNA sequence. Cas polypeptides include naturally occurring Cas proteins and engineered, altered, or otherwise modified Cas proteins that differ by one or more amino acid residues from a naturally-occurring Cas sequence.

A guide RNA (gRNA) comprises two segments, a DNA-binding segment and a protein-binding segment. In some aspects, the protein-binding segment of a gRNA is comprised in one RNA molecule and the DNA-binding segment is comprised in another separate RNA molecule. Such aspects are referred to herein as “double-molecule gRNAs” or “two-molecule gRNA” or “dual gRNAs.” In some aspects, the gRNA is a single RNA molecule and is referred to herein as a “single-guide RNA” or an “sgRNA.” The term “guide RNA” or “gRNA” is inclusive, referring both to two-molecule guide RNAs and sgRNAs.

The protein-binding segment of a gRNA comprises, in part, two complementary stretches of nucleotides that hybridize to one another to form a double stranded RNA duplex (dsRNA duplex), which facilitates binding to the Cas protein. The nucleic acid-binding segment (or “nucleic acid-binding sequence”) of a gRNA comprises a nucleotide sequence that is complementary to and capable of binding to a specific target nucleic acid sequence. The protein-binding segment of the gRNA interacts with a Cas polypeptide and the interaction of the gRNA molecule and site-directed modifying polypeptide results in Cas binding to the endogenous nucleic acid sequence and produces one or more modifications within or around the target nucleic acid sequence. The precise location of the target modification site is determined by both (i) base-pairing complementarity between the gRNA and the target nucleic acid sequence; and (ii) the location of a short motif, referred to as the protospacer adjacent motif (PAM), in the target DNA sequence (referred to as a protospacer flanking sequence (PFS) in target RNA sequences). The PAM/PFS sequence is required for Cas binding to the target nucleic acid sequence. A variety of PAM/PFS sequences are known in the art and are suitable for use with a particular Cas endonuclease (e.g., a Cas9 endonuclease) (See e.g., Nat Methods. 2013 November; 10(11): 1116-1121 and Sci Rep. 2014; 4: 5405). In some aspects, the PAM sequence is located within 50 base pairs of the target modification site in a target DNA sequence. In some aspects, the PAM sequence is located within 10 base pairs of the target modification site in a target DNA sequence. The DNA sequences that can be targeted by this method are limited only by the relative distance of the PAM sequence to the target modification site and the presence of a unique 20 base pair sequence to mediate sequence-specific, gRNA-mediated Cas binding. In some aspects, the PFS sequence is located at the 3′ end of the target RNA sequence. In some aspects, the target modification site is located at the 5′ terminus of the target locus. In some aspects, the target modification site is located at the 3′ end of the target locus. In some aspects, the target modification site is located within an intron or an exon of the target locus.

In some aspects, the present disclosure provides a polynucleotide encoding a gRNA. In some aspects, a gRNA-encoding nucleic acid is comprised in an expression vector, e.g., a recombinant expression vector. In some aspects, the present disclosure provides a polynucleotide encoding a site-directed modifying polypeptide. In some aspects, the polynucleotide encoding a site-directed modifying polypeptide is comprised in an expression vector, e.g., a recombinant expression vector.

### 1. Cas Proteins

In some aspects, the site-directed modifying polypeptide is a Cas protein. Any Cas protein, including those provided herein, can be used. Cas molecules of a variety of species can be used in the methods and compositions described herein, including Cas molecules derived from S. pyogenes, S. aureus, N. meningitidis, S. thermophiles, Acidovorax avenae, Actinobacillus pleuropneumonias, Actinobacillus succinogenes, Actinobacillus suis, Actinomyces sp., Cychphilusdenitrificans, Aminomonas paucivorans, Bacillus cereus, Bacillus smithii, Bacillus thuringiensis, Bacteroides sp., Blastopirellula marina, Bradyrhizobium sp., Brevibacillus laterospoxus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteobacterium, Gluconacetobacter diazotrophicus, Haemophilus parainfluenzae, Haemophilus sputomm, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., or Verminephrobacter eiseniae.

In some aspects, the Cas protein is a naturally-occurring Cas protein. In some aspects, the Cas endonuclease is selected from the group consisting of C2C1, C2C3, Cpf1 (also referred to as Cas12a), Cas12b, Cas12c, Cas12d, Cas12e, Cas13a, Cas13b, Cas13c, Cas13d, Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1 , Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

In some aspects, the Cas protein is an endoribonuclease such as a Cas13 protein. In some aspects, the Cas13 protein is a Cas13a (Abudayyeh et al., Nature 550 (2017), 280-284), Cas13b (Cox et al., Science (2017) 358:6336, 1019-1027), Cas13c (Cox et al., Science (2017) 358:6336, 1019-1027), or Cas13d (Zhang et al., Cell 175 (2018), 212-223) protein.

In some aspects, the Cas9 protein is any Cas9 protein, including any of the Cas9 proteins specifically provided herein. In some aspects, the Cas protein is a wild-type or naturally occurring Cas9 protein or a Cas9 ortholog. Wild-type Cas9 is a multi-domain enzyme that uses an HNH nuclease domain to cleave the target strand of DNA and a RuvC-like domain to cleave the non-target strand. Binding of WT Cas9 to DNA based on gRNA specificity results in double-stranded DNA breaks that can be repaired by non-homologous end joining (NHEJ) or homology-directed repair (HDR). Exemplary naturally occurring Cas9 molecules are described in Chylinski et al., RNA Biology 2013 10:5, 727-737 and additional Cas9 orthologs are described in International PCT Publication No. WO 2015/071474. Such Cas9 molecules include Cas9 molecules of a cluster 1 bacterial family, cluster 2 bacterial family, cluster 3 bacterial family, cluster 4 bacterial family, cluster 5 bacterial family, cluster 6 bacterial family, a cluster 7 bacterial family, a cluster 8 bacterial family, a cluster 9 bacterial family, a cluster 10 bacterial family, a cluster 1 1 bacterial family, a cluster 12 bacterial family, a cluster 13 bacterial family, a cluster 14 bacterial family, a cluster 15 bacterial family, a cluster 16 bacterial family, a cluster 17 bacterial family, a cluster 18 bacterial family, a cluster 19 bacterial family, a cluster 20 bacterial family, a cluster 21 bacterial family, a cluster 22 bacterial family, a cluster 23 bacterial family, a cluster 24 bacterial family, a cluster 25 bacterial family, a cluster 26 bacterial family, a cluster 27 bacterial family, a cluster 28 bacterial family, a cluster 29 bacterial family, a cluster 30 bacterial family, a cluster 31 bacterial family, a cluster 32 bacterial family, a cluster 33 bacterial family, a cluster 34 bacterial family, a cluster 35 bacterial family, a cluster 36 bacterial family, a cluster 37 bacterial family, a cluster 38 bacterial family, a cluster 39 bacterial family, a cluster 40 bacterial family, a cluster 41 bacterial family, a cluster 42 bacterial family, a cluster 43 bacterial family, a cluster 44 bacterial family, a cluster 45 bacterial family, a cluster 46 bacterial family, a cluster 47 bacterial family, a cluster 48 bacterial family, a cluster 49 bacterial family, a cluster 50 bacterial family, a cluster 51 bacterial family, a cluster 52 bacterial family, a cluster 53 bacterial family, a cluster 54 bacterial family, a cluster 55 bacterial family, a cluster 56 bacterial family, a cluster 57 bacterial family, a cluster 58 bacterial family, a cluster 59 bacterial family, a cluster 60 bacterial family, a cluster 61 bacterial family, a cluster 62 bacterial family, a cluster 63 bacterial family, a cluster 64 bacterial family, a cluster 65 bacterial family, a cluster 66 bacterial family, a cluster 67 bacterial family, a cluster 68 bacterial family, a cluster 69 bacterial family, a cluster 70 bacterial family, a cluster 71 bacterial family, a cluster 72 bacterial family, a cluster 73 bacterial family, a cluster 74 bacterial family, a cluster 75 bacterial family, a cluster 76 bacterial family, a cluster 77 bacterial family, or a cluster 78 bacterial family.

In some aspects, the naturally occurring Cas9 polypeptide is selected from the group consisting of SpCas9, SpCas9-HF1, SpCas9-HF2, SpCas9-HF3, SpCas9-HF4, SaCas9, FnCpf, FnCas9, eSpCas9, and NmeCas9. In some aspects, the Cas9 protein comprises an amino acid sequence having at least 60%, 65%, 70%, 75%, 80%, 85%, 90%, 95%, 96%, 97%, 98%, 99%, or 100% sequence identity to a Cas9 amino acid sequence described in Chylinski et al., RNA Biology 2013 10:5, 727-737; Hou et al., PNAS Early Edition 2013, 1-6).

In some aspects, the Cas polypeptide comprises one or more of the following activities:
a. a nickase activity, i.e., the ability to cleave a single strand, e.g., the non-complementary strand or the complementary strand, of a nucleic acid molecule;
b. a double stranded nuclease activity, i.e., the ability to cleave both strands of a double stranded nucleic acid and create a double stranded break, which in an aspect is the presence of two nickase activities;
c. an endonuclease activity;
d. an exonuclease activity; and/or
e. a helicase activity, i.e., the ability to unwind the helical structure of a double stranded nucleic acid.

In some aspects, the Cas polypeptide is fused to heterologous proteins that recruit DNA-damage signaling proteins, exonucleases, or phosphatases to further increase the likelihood or the rate of repair of the target sequence by one repair mechanism or another. In some aspects, a WT Cas polypeptide is co-expressed with a nucleic acid repair template to facilitate the incorporation of an exogenous nucleic acid sequence by homology-directed repair.

In some aspects, different Cas proteins (i.e., Cas9 proteins from various species) may be advantageous to use in the various provided methods in order to capitalize on various enzymatic characteristics of the different Cas proteins (e.g., for different PAM sequence preferences; for increased or decreased enzymatic activity; for an increased or decreased level of cellular toxicity; to change the balance between NHEJ, homology-directed repair, single strand breaks, double strand breaks, etc.).

In some aspects, the Cas protein is a Cas9 protein derived from S. pyogenes and recognizes the PAM sequence motif NGG, NAG, NGA (Mali et al, Science 2013; 339(6121): 823-826). In some aspects, the Cas protein is a Cas9 protein derived from S. thermophiles and recognizes the PAM sequence motif NGGNG and/or NNAGAAW (W=A or T) (See, e.g., Horvath et al, Science, 2010; 327(5962): 167-170, and Deveau et al, J Bacteriol 2008; 190(4): 1390-1400). In some aspects, the Cas protein is a Cas9 protein derived from S. mutans and recognizes the PAM sequence motif NGG and/or NAAR (R=A or G) (See, e.g., Deveau et al, J BACTERIOL 2008; 190(4): 1390-1400). In some aspects, the Cas protein is a Cas9 protein derived from S. aureus and recognizes the PAM sequence motif NNGRR (R=A or G). In some aspects, the Cas protein is a Cas9 protein derived from S. aureus and recognizes the PAM sequence motif N GRRT (R=A or G). In some aspects, the Cas protein is a Cas9 protein derived from S. aureus and recognizes the PAM sequence motif N GRRV (R=A or G). In some aspects, the Cas protein is a Cas9 protein derived from N. meningitidis and recognizes the PAM sequence motif N GATT or N GCTT (R=A or G, V=A, G or C) (See, e.g., Hou et ah, PNAS 2013, 1-6). In the aforementioned aspects, N can be any nucleotide residue, e.g., any of A, G, C or T. In some aspects, the Cas protein is a Cas13a protein derived from Leptotrichia shahii and recognizes the PFS sequence motif of a single 3′ A, U, or C.

In some aspects, a polynucleotide encoding a Cas protein is provided. In some aspects, the polynucleotide encodes a Cas protein that is at least 90% identical to a Cas protein described in International PCT Publication No. WO 2015/071474 or Chylinski et al., RNA Biology 2013 10:5, 727-737. In some aspects, the polynucleotide encodes a Cas protein that is at least 95%, 96%, 97%, 98%, or 99% identical to a Cas protein described in International PCT Publication No. WO 2015/071474 or Chylinski et al., RNA Biology 2013 10:5, 727-737. In some aspects, the polynucleotide encodes a Cas protein that is 100% identical to a Cas protein described in International PCT Publication No. WO 2015/071474 or Chylinski et al., RNA Biology 2013 10:5, 727-737.

### 2. Cas Mutants

In some aspects, the Cas polypeptides are engineered to alter one or more properties of the Cas polypeptide. For example, in some aspects, the Cas polypeptide comprises altered enzymatic properties, e.g., altered nuclease activity, (as compared with a naturally occurring or other reference Cas molecule) or altered helicase activity. In some aspects, an engineered Cas polypeptide can have an alteration that alters its size, e.g., a deletion of amino acid sequence that reduces its size without significant effect on another property of the Cas polypeptide. In some aspects, an engineered Cas polypeptide comprises an alteration that affects PAM recognition. For example, an engineered Cas polypeptide can be altered to recognize a PAM sequence other than the PAM sequence recognized by the corresponding wild-type Cas protein.

Cas polypeptides with desired properties can be made in a number of ways, including alteration of a naturally occurring Cas polypeptide or parental Cas polypeptide, to provide a mutant or altered Cas polypeptide having a desired property. For example, one or more mutations can be introduced into the sequence of a parental Cas polypeptide (e.g., a naturally occurring or engineered Cas polypeptide). Such mutations and differences may comprise substitutions (e.g., conservative substitutions or substitutions of non-essential amino acids); insertions; or deletions. In some aspects, a mutant Cas polypeptide comprises one or more mutations (e.g., at least 1, 2, 3, 4, 5, 10, 15, 20, 30, 40 or 50 mutations) relative to a parental Cas polypeptide.

In some aspects, a mutant Cas polypeptide comprises a cleavage property that differs from a naturally occurring Cas polypeptide. In some aspects, the Cas is a deactivated Cas (dCas) mutant. In some aspects, the Cas polypeptide does not comprise any intrinsic enzymatic activity and is unable to mediate target nucleic acid cleavage. In some aspects, the dCas may be fused with a heterologous protein that is capable of modifying the target nucleic acid in a non-cleavage based manner. For example, in some aspects, a dCas protein is fused to transcription activator or transcription repressor domains (e.g., the Kruppel associated box (KRAB or SKD); the Mad mSIN3 interaction domain (SID or SID4X); the ERF repressor domain (ERD); the MAX-interacting protein 1 (MXI1); methyl-CpG binding protein 2 (MECP2); etc.). In some such cases, the dCas fusion protein is targeted by the gRNA to a specific location (i.e., sequence) in the target nucleic acid and exerts locus-specific regulation such as blocking RNA polymerase binding to a promoter (which selectively inhibits transcription activator function), and/or modifying the local chromatin status (e.g., when a fusion sequence is used that modifies the target DNA or modifies a polypeptide associated with the target DNA). In some cases, the changes are transient (e.g., transcription repression or activation). In some cases, the changes are inheritable (e.g., when epigenetic modifications are made to the target DNA or to proteins associated with the target DNA, e.g., nucleosomal histones).

In some aspects, the dCas is a dCas13 mutant (Konermann et al., Cell 173 (2018), 665-676). These dCas13 mutants can then be fused to enzymes that modify RNA, including adenosine deaminases (e.g., ADAR1 and ADAR2). Adenosine deaminases convert adenine to inosine, which the translational machinery treats like guanine, thereby creating a functional A→G change in the RNA sequence. In some aspects, the dCas is a dCas9 mutant.

In some aspects, the mutant Cas9 is a Cas9 nickase mutant. Cas9 nickase mutants

comprise only one catalytically active domain (either the HNH domain or the RuvC domain). The Cas9 nickase mutants retain DNA binding based on gRNA specificity, but are capable of cutting only one strand of DNA resulting in a single-strand break (e.g. a “nick”). In some aspects, two complementary Cas9 nickase mutants (e.g., one Cas9 nickase mutant with an inactivated RuvC domain, and one Cas9 nickase mutant with an inactivated HNH domain) are expressed in the same cell with two gRNAs corresponding to two respective target sequences; one target sequence on the sense DNA strand, and one on the antisense DNA strand. This dual-nickase system results in staggered double stranded breaks and can increase target specificity, as it is unlikely that two off-target nicks will be generated close enough to generate a double stranded break. In some aspects, a Cas9 nickase mutant is co-expressed with a nucleic acid repair template to facilitate the incorporation of an exogenous nucleic acid sequence by homology-directed repair.

In some aspects, the Cas polypeptides described herein can be engineered to alter the PAM/PFS specificity of the Cas polypeptide. In some aspects, a mutant Cas polypeptide has a PAM/PFS specificity that is different from the PAM/PFS specificity of the parental Cas polypeptide. For example, a naturally occurring Cas protein can be modified to alter the PAM/PFS sequence that the mutant Cas polypeptide recognizes to decrease off target sites, improve specificity, or eliminate a PAM/PFS recognition requirement. In some aspects, a Cas protein can be modified to increase the length of the PAM/PFS recognition sequence. In some aspects, the length of the PAM recognition sequence is at least 4, 5, 6, 7, 8, 9, 10 or 15 amino acids in length. Cas polypeptides that recognize different PAM/PFS sequences and/or have reduced off-target activity can be generated using directed evolution. Exemplary methods and systems that can be used for directed evolution of Cas polypeptides are described, e.g., in Esvelt et al. Nature 2011, 472(7344): 499-503.

Exemplary Cas mutants are described in International PCT Publication No. WO 2015/161276 and Konermann et al., Cell 173 (2018), 665-676, which are incorporated herein by reference in their entireties.

3. gRNAs

The present disclosure provides guide RNAs (gRNAs) that direct a site-directed modifying polypeptide to a specific target nucleic acid sequence. A gRNA comprises a nucleic acid-targeting segment and protein-binding segment. The nucleic acid-targeting segment of a gRNA comprises a nucleotide sequence that is complementary to a sequence in the target nucleic acid sequence. As such, the nucleic acid-targeting segment of a gRNA interacts with a target nucleic acid in a sequence-specific manner via hybridization (i.e., base pairing), and the nucleotide sequence of the nucleic acid-targeting segment determines the location within the target nucleic acid that the gRNA will bind. The nucleic acid-targeting segment of a gRNA can be modified (e.g., by genetic engineering) to hybridize to any desired sequence within a target nucleic acid sequence.

The protein-binding segment of a guide RNA interacts with a site-directed modifying polypeptide (e.g. a Cas protein) to form a complex. The guide RNA guides the bound polypeptide to a specific nucleotide sequence within target nucleic acid via the above-described nucleic acid-targeting segment. The protein-binding segment of a guide RNA comprises two stretches of nucleotides that are complementary to one another and which form a double stranded RNA duplex.

In some aspects, a gRNA comprises two separate RNA molecules. In some aspects, each of the two RNA molecules comprises a stretch of nucleotides that are complementary to one another such that the complementary nucleotides of the two RNA molecules hybridize to form the double-stranded RNA duplex of the protein-binding segment. In some aspects, a gRNA comprises a single RNA molecule (sgRNA).

The specificity of a gRNA for a target loci is mediated by the sequence of the nucleic acid-binding segment, which comprises about 20 nucleotides that are complementary to a target nucleic acid sequence within the target locus. In some aspects, the corresponding target nucleic acid sequence is approximately 20 nucleotides in length. In some aspects, the nucleic acid-binding segments of the gRNA sequences of the present disclosure are at least 90% complementary to a target nucleic acid sequence within a target locus. In some aspects, the nucleic acid-binding segments of the gRNA sequences of the present disclosure are at least 95%, 96%, 97%, 98%, or 99% complementary to a target nucleic acid sequence within a target locus. In some aspects, the nucleic acid-binding segments of the gRNA sequences of the present disclosure are 100% complementary to a target nucleic acid sequence within a target locus.

In some aspects, the target nucleic acid sequence is an RNA target sequence. In some aspects, the target nucleic acid sequence is a DNA target sequence. In some aspects, the nucleic acid-binding segments of the gRNA sequences bind to a target DNA sequence that is at least 90% identical to a target DNA sequence of a target gene selected from those listed in Table 1. In some aspects, the nucleic acid-binding segments of the gRNA sequences bind to a target DNA sequence that is at least 95%, 96%, 97%, 98%, or 99% identical to a target DNA sequence of a target gene selected from those listed in Table 1. In some aspects, the nucleic acid-binding segments of the gRNA sequences bind to a target DNA sequence that is 100% identical to a target DNA sequence of a target gene selected from those listed in Table 1.

In some aspects, the gene-regulating system comprises two or more gRNA molecules each comprising a DNA-binding segment, wherein at least one of the nucleic acid-binding segments binds to a target DNA sequence that is at least 90% identical to a target DNA sequence of a target gene selected from Table 1. In some aspects, the gene-regulating system comprises two or more gRNA molecules each comprising a nucleic acid-binding segment, wherein at least one of the nucleic acid-binding segments binds to a target DNA sequence that is at least 95%, 96%, 97%, 98%, or 99% identical to a target DNA sequence of a target gene selected from Table 1. In some aspects, the gene-regulating system comprises two or more gRNA molecules each comprising a nucleic acid-binding segment, wherein at least one of the nucleic acid-binding segments binds to a target DNA sequence that is 100% to a target DNA sequence of a target gene selected from Table 1.

In some aspects, the nucleic acid-binding segments of the gRNA sequences

described herein are designed to minimize off-target binding using algorithms known in the art (e.g., Cas-OFF finder) to identify target sequences that are unique to a particular target locus or target gene.

In some aspects, the gRNAs described herein can comprise one or more modified nucleosides or nucleotides which introduce stability toward nucleases. In some aspects, these modified gRNAs may elicit a reduced innate immune response as compared to a non-modified gRNA. The term “innate immune response” includes a cellular response to exogenous nucleic acids, including single stranded nucleic acids, generally of viral or bacterial origin, which involves the induction of cytokine expression and release, particularly the interferons, and cell death.

In some aspects, the gRNAs described herein are modified at or near the 5′ end (e.g., within 1-10, 1-5, or 1-2 nucleotides of their 5′ end). In some aspects, the 5′ end of a gRNA is modified by the inclusion of a eukaryotic mRNA cap structure or cap analog (e.g., a G(5′)ppp(5′)G cap analog, a m7G(5′)ppp(5′)G cap analog, or a 3′-0-Me-m7G(5′)ppp(5′)G anti reverse cap analog (ARCA)). In some aspects, an in vitro transcribed gRNA is modified by treatment with a phosphatase (e.g., calf intestinal alkaline phosphatase) to remove the 5′ triphosphate group. In some aspects, a gRNA comprises a modification at or near its 3′ end (e.g., within 1-10, 1-5, or 1-2 nucleotides of its 3′ end). For example, in some aspects, the 3′ end of a gRNA is modified by the addition of one or more (e.g., 25-200) adenine (A) residues.

In some aspects, modified nucleosides and modified nucleotides can be present in a gRNA, but also may be present in other gene-regulating systems, e.g., mRNA, RNAi, or siRNA-based systems. In some aspects, modified nucleosides and nucleotides can include one or more of:
a. alteration, e.g., replacement, of one or both of the non-linking phosphate oxygens and/or of one or more of the linking phosphate oxygens in the phosphodiester backbone linkage;
b. alteration, e.g., replacement, of a constituent of the ribose sugar, e.g., of the 2′ hydroxyl on the ribose sugar;
c. wholesale replacement of the phosphate moiety with “dephospho” linkers;
d. modification or replacement of a naturally occurring nucleobase;
e. replacement or modification of the ribose-phosphate backbone;
f. modification of the 3′ end or 5′ end of the oligonucleotide, e.g., removal, modification or replacement of a terminal phosphate group or conjugation of a moiety; and
g. modification of the sugar.

In some aspects, the modifications listed above can be combined to provide modified nucleosides and nucleotides that can have two, three, four, or more modifications. For example, in some aspects, a modified nucleoside or nucleotide can have a modified sugar and a modified nucleobase. In some aspects, every base of a gRNA is modified. In some aspects, each of the phosphate groups of a gRNA molecule are replaced with phosphorothioate groups.

In some aspects, a software tool can be used to optimize the choice of gRNA within a user's target sequence, e.g., to minimize total off-target activity across the genome. Off target activity may be other than cleavage. For example, for each possible gRNA choice using S. pyogenes Cas9, software tools can identify all potential off-target sequences (preceding either NAG or NGG PAMs) across the genome that contain up to a certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible gRNA can then be ranked according to its total predicted off-target cleavage; the top-ranked gRNAs represent those that are likely to have the greatest on-target and the least off-target cleavage. Other functions, e.g., automated reagent design for gRNA vector construction, primer design for the on-target Surveyor assay, and primer design for high-throughput detection and quantification of off-target cleavage via next-generation sequencing, can also be included in the tool.

Some methods provided herein begin with genetic engineering. Such genetic engineering can be followed e.g., by one or more resting steps and/or one or more stimulating steps. For example, Tregs can be genetically engineered, rested, and then stimulated. Alternatively, Tregs can be genetically engineered, stimulated, and then rested.

In other aspects of methods provided herein, Tregs can be genetically engineered after one or more resting steps and/or one or more stimulating steps. For example, Tregs can be genetically engineered after a first stimulating step, a resting step, and a second stimulating step.

In some aspects, Tregs are cultured in the presence of a stimulatory agent immediately prior to the genetic engineering. In some aspects, the Tregs are genetically engineered in the absence of a stimulatory agent. In some aspects, the Tregs are cultured in the absence of a stimulatory agent (rested) immediately after genetic engineering. In some aspects, Tregs are genetically engineered during a resting step (e.g., in the absence of a stimulatory agent).

In some aspects, Tregs are cultured in the absence of a stimulatory agent immediately prior to the genetic engineering.

In some aspects, Tregs are genetically engineered after about 6 days to about 12 days in culture. In some aspects, Tregs are genetically engineered after about 6 days to about 11 days in culture. In some aspects, Tregs are genetically engineered after about 6 days to about 10 days in culture. In some aspects, Tregs are genetically engineered after about 6 days to about 9 days in culture. In some aspects, Tregs are genetically engineered after about 6 days to about 8 days in culture. In some aspects, Tregs are genetically engineered after about 7 days to about 12 days in culture. In some aspects, Tregs are genetically engineered after about 7 days to about 11 days in culture. In some aspects, Tregs are genetically engineered after about 7 days to about 10 days in culture. In some aspects, Tregs are genetically engineered after about 7 days to about 9 days in culture. In some aspects, Tregs are genetically engineered after about 7 days to about 8 days in culture.

In some aspects, Tregs are genetically engineered after at least 6 days in culture. In some aspects, Tregs are genetically engineered after at least 7 days in culture. In some aspects, Tregs are genetically engineered after at least 8 days in culture.

In some aspects, Tregs are genetically engineered after about 6 days in culture. In some aspects, Tregs are genetically engineered after about 7 days in culture. In some aspects, Tregs are genetically engineered after about 8 days in culture. In some aspects, Tregs are genetically engineered after about 9 days in culture. In some aspects, Tregs are genetically engineered after about 10 days in culture.

In some aspects, Tregs are genetically engineered after no more than 10 total days of exposure to a stimulating agent, wherein the 10 total days do not include more than 5 consecutive days. In some aspects, Tregs are genetically engineered after no more than 9 total days of exposure to a stimulating agent, wherein the 9 total days do not include more than 5 consecutive days. In some aspects, Tregs are genetically engineered after no more than 8 total days of exposure to a stimulating agent, wherein the 8 total days do not include more than 5 consecutive days. In some aspects, Tregs are genetically engineered after no more than 7 total days of exposure to a stimulating agent, wherein the 7 total days do not include more than 5 consecutive days. In some aspects, Tregs are genetically engineered after no more than 6 total days of exposure to a stimulating agent, wherein the 6 total days do not include more than 5 consecutive days.

In some aspects, Tregs are genetically engineered after at least 4 total days of exposure to a stimulating agent, wherein the 4 total days do not include more than 3 consecutive days. In some aspects, Tregs are genetically engineered after at least 5 total days of exposure to a stimulating agent, wherein the 5 total days do not include more than 3 consecutive days. In some aspects, Tregs are genetically engineered after at least 6 total days of exposure to a stimulating agent, wherein the 4 total days do not include more than 6 consecutive days. In some aspects, Tregs are genetically engineered after at least 7 total days of exposure to a stimulating agent, wherein the 7 total days do not include more than 5 consecutive days.

The days in culture prior to genetic engineering can comprise, e.g., a first stimulating step, a resting step, and a second stimulating step.

In some aspects, Tregs are genetically engineered when the population of Tregs has expanded about 100-fold to about 500-fold. In some aspects, Tregs are genetically engineered when the population of Tregs has expanded about 250-fold.

### Isolation of Tregs

Prior to expansion, Tregs can be isolated, e.g., from a subject or a sample obtained from a subject. In some aspects, Tregs are obtained from a subject's peripheral blood, thymus, lymph nodes, spleen, bone marrow, umbilical cord blood, or tissue sample. In some aspects, Tregs are obtained from a sample obtained from a subject's peripheral blood, thymus, lymph nodes, spleen, bone marrow, umbilical cord blood, or tissue sample. The subject can be, for example, a mammalian subject such as a human subject.

In some aspects, Tregs are obtained from peripheral blood. In some aspects, Tregs are obtained from human peripheral blood.

In some aspects Tregs are obtained from PBMCs from human peripheral blood, wherein CD4⁺ T cells are isolated via negative immunomagnetic selection (e.g., using EasySep Human CD4⁺ T Cell Isolation Kit), CD4⁺ T cells are labeled with antibodies to CD25, CD4, CD127, and hCD45RA, and CD4⁺CD25^{hi}CD127^{lo}CD45RA⁺ Tregs are selected.

PBMCs can be isolated from blood by density gradient sedimentation, and CD4⁺ T cells can be enriched by positive selection from PBMCs by magnetic cell sorting. CD4⁺ T cells can then be stained with flurochrome-labeled antibodies specific regulatory T cell markers such as CD4, CD25, and/or CD127, and then separated by fluorescence-activated cell sorting (FACS) to enrich CD4⁺CD25⁺CD127^{lo} Tregs and separate them from, e.g., CD4⁺CD25⁻CD127⁺ conventional T cells.

Once a sample containing Tregs is isolated, Tregs can be enriched. Tregs can be enriched, for example, by targeting for selection of cell surface markers specific for immune suppressive Tregs and separating using automated cell sorting such as FACS, solid-phase magnetic beads, etc. Methods of enriching Tregs are provided, for example, in U.S. Pat. No. 7,722,862, which is herein incorporated by reference in its entirety. Enrichment can comprise positive selection and/or negative selection. For example, negative selection can be used to remove cells with surface markers specific to non-Treg cell types such as CD8, CD11b, CD16, CD19, CD36 and/or CD56.

In some aspects, Tregs are isolated using cell sorting for CD45RA^{hi}CD25^{hi}CD127^{lo}CD4⁺ cells, e.g., using FACS, of CD4+ T cells. In some aspects, Tregs are isolated using cell sorting for total CD45RA^{hi}CD25^{hi}CD127^{lo}CD4⁺ cells.

In some aspects, Tregs are isolated by enriching CD25+ T cells using magnetic beads. In some aspects, Tregs are isolated by enriching CD25+ T cells using Fluorescent Activated Cell Sorting (FACS).

In some aspects, Tregs are isolated based on the presence of CD4 and CD25, and a lack of the α-chain of IL-7R (CD127).

In some aspects, Tregs are isolated without using magnetic beads. In some aspects, Tregs are isolated without using artificial antigen presenting cells. In some aspects, Tregs are isolated without using magnetic beads or artificial antigen presenting cells.

In some aspects, Tregs are obtained by isolating peripheral blood mononuclear cells (PBMC) from a subject using lymphocyte density gradient centrifugation, depleting CD8+ cells, and then enriching CD25+ T cells using magnetic beads.

In some aspects, Tregs are isolated (e.g., from a subject) by bead separation.

### Uses of Expanded Tregs

Also provided herein are methods of using Tregs that have been expanded according to the methods provided herein. For example, the expanded Tregs can be administered to a subject. The administration can be, e.g., for treating an autoimmune or inflammatory disease in a subject, for treating or preventing graft vs. host disease (GVHD) in a subject, and/or for decreasing an immune response in a subject. The subject can be a mammalian subject, e.g., a human subject.

In some aspects, the autoimmune or inflammatory disease or disorder is selected from the group consisting of: psoriasis, systemic lupus erythematosus, rheumatoid arthritis, type I diabetes, amyotrophic lateral sclerosis (ALS), multiple sclerosis, ulcerative colitis, Crohn's disease, HCV-related vasculitis, alopecia areata, ankylosing spondylitis, Sjogren's Syndrome, autoimmune hepatitis, inflammatory bowel disease (IBD), colitis, vasculitis, temporal arthritis, lupus, celiac disease, polymyalgia rheumatic, and arthritis.

In some aspects, Tregs are obtained from a subject, expanded according to any method provided herein, and then administered to the same subject. Accordingly, use of the expanded Tregs provided herein can be autologous.

In some aspects, Tregs are obtained from a subject, expanded according to any method provided herein, and then administered to a different subject. Accordingly, use of the expanded Tregs provided herein can be allogenic.

The amount of Tregs that will be effective in the treatment or prevention of a condition will depend on the nature of the disease. The precise dose to be employed will also depend on the route of administration and the seriousness of the condition. Typically, administration of T cell therapies is defined by number of cells per kilogram of body weight. However, T cells replicate and expand after administration. The cells can be administered by infusion techniques that are known in the art.

### EXAMPLES

The experiments described herein use a cyclical in vitro approach to maximize the growth of human regulatory T cells (Treg) for their clinical use as an immunotherapy for the treatment of autoimmune disease.

### Example 1

### Materials and Methods

### Materials

gRNAs: Where indicated, all experiments use single-molecule gRNAs (sgRNAs). Dual gRNA molecules were formed by duplexing 20011M tracrRNA (IDT Cat #1072534) with 20011M of target-specific crRNA (IDT) in nuclease free duplex buffer (IDT Cat #11-01-03-01) for 5 min at 95° C., to form 100 μM of tracrRNA:crRNA duplex, where the tracrRNA and crRNA are present at a 1:1 ratio.

Cas9: Cas9 was expressed in target cells by introduction of Cas9 protein derived from S. pyogenes (IDT Cat #1074182).

RNPs: gRNA-Cas9 ribonucleoproteins (RNPs) were formed by combining 1.2 μL of 100 μM tracrRNA:crRNA duplex with 1 μL of 20 μM Cas9 protein and 0.8 μL of PBS. Mixtures were incubated at RT for 20 minutes to form the RNP complexes.

### Methods

Human Treg cell Isolation: Peripheral blood Treg cells were isolated from fresh leukopacks or whole blood from healthy volunteer blood donors in a step-wise fashion. First, peripheral blood mononuclear cells (PBMCs) were isolated using the EasySep Direct Human PBMC Isolation Kit (StemCell Technologies, Cat #19654). Next, CD4⁺ T cells were isolated via negative immunomagnetic selection using EasySep Human CD4⁺ T Cell Isolation Kit (StemCell Technologies, Cat #17952). Isolated CD4⁺ T cells were labeled with antibodies to hCD25 (PE, BioLegend), hCD4 (APC, BD Pharmingen), hCD127 (BV421, BD Pharmingen) and hCD45RA (APC-Cy7, BD Pharmingen), and then sorted based on the following parameters: CD4⁺CD25^{hi}CD127^{lo}CD45RA⁺.

Human Treg cell expansion ex vivo: Isolated Tregs were plated at 1.25×10⁵ cells/mL (0.2 mL per well) in X-VIVO 15 T Cell Expansion Medium (Lonza, Cat #04-418Q) supplemented with 10% human inactivated serum (hereafter referred to as Treg media), human IL-2 (800 units/mL), N-Acetyl-L-cysteine (5 mM) and 10 μl/mL ImmunoCult CD3/28/2 tetramer (StemCell Technologies, Cat #10970) in 96 well u-bottom plates (Falcon, BD Pharmingen). On day 3 of culture, cells were washed, re-plated at the original concentration in Treg media containing IL-2 and then allowed to expand for 3 days. During this expansion phase, cells were split daily to maintain a concentration of 1.25×10⁵ cells/mL. On day 6 of culture, cells were re-stimulated with CD3/28/2 tetramers (10 μl/mL) and the concentration was adjusted to 5×10⁵ cells/mL (this cell density was maintained for the remainder of the culture period). Concentrations of IL-2 were maintained at 800 units/mL during the first 7 days of culture and thereafter reduced to 300 units/mL. Tetramers were only added to the cultures on the first day of each stimulation cycle and were not replenished daily. Cycles of stimulation and expansion were repeated every 3-4 days throughout the remainder of the culture period. The purities of cultured Treg cells were checked periodically by flow cytometry as outlined below.

RNP transfection of Treg cells: Where indicated, Tregs were edited using Cas9-RNPs as follows: On day 8 of culture, Treg cells were washed and resuspended in PBS at 5×10⁷ cells/mL. A single guide RNA (sgRNA) targeting a for Treg cells irrelevant gene, OR1A1 (SEQ ID NO:7 GCTGACCAGTAACTCCCAGG), was complexed with trans-activating CRISPR RNA (tracrRNA) in vitro for 5 min at 95° C. The newly generated duplex molecules were mixed with Cas9 protein as described in Materials (supra) and incubated at room-temperature for 20 min to form ribonucleoproteins (RNPs). Immediately prior to electroporation, cells were resuspended in nucleofection buffer (18% supplement 1, 82% P3 buffer from the Amaxa P3 primary cell 4D-Nuclefector X kit S (Cat #V4XP-3032)) at a concentration of 8×10⁷ cells/mL. This cell suspension was combined with the RNP solution form the step above and an inert single-stranded DNA oligonucleotide (Alt-R Cas9 Electroporation Enhancer) at a ratio of 20:5:1. Cells were electroporated following the “T cell, Human, Stim” program (EO-115). After electroporation, 80 μL of warm X-VIVO 15 media was added to each well, and cells were pooled into a culture flask at a density of 2×10⁶ cells/mL in X-VIVO 15 media containing IL-2 (300 units/mL).

Assessment of editing efficiency. For this method, genomic DNA (gDNA) was isolated from edited T cells using the Qiagen Blood and Cell Culture DNA Mini Kit (Cat #: 13323) following the vendor recommended protocol and quantified. Following gDNA isolation, PCR was performed to amplify the region of edited genomic DNA using locus-specific PCR primers containing overhangs required for the addition of Illumina Next Generation sequencing adapters. The resulting PCR product was run on a 1% agarose gel to ensure specific and adequate amplification of the genomic locus occurred before PCR cleanup was conducted according to the vendor recommended protocol using the Monarch PCR & DNA Cleanup Kit (Cat #: T1030S). Purified PCR product was then quantified, and a second PCR was performed to anneal the Illumina sequencing adapters and sample specific indexing sequences required for multiplexing. Following this, the PCR product was run on a 1% agarose gel to assess size before being purified using AMPure XP beads (produced internally). Purified PCR product was then quantified via qPCR using the Kapa Illumina Library Quantification Kit (Cat #: KK4923) and Kapa Illumina Library Quantification DNA Standards (Cat #: KK4903). Quantified product was then loaded on the Illumina NextSeq 500 system using the Illumina NextSeq 500/550 Mid Output Reagent Cartridge v2 (Cat #: FC-404-2003). Analysis of produced sequencing data was performed to assess insertions and deletions (indels) at the anticipated cut site in the DNA of the edited T cell pool.

Immunophenotyping and TSDR analysis of OR1A1-edited Treg cells: The purity of cultured Treg cells was examined periodically during expansion by surface staining for CD4 (clone SK3, 10 min at room-temperature), followed by viability dye (LIVE/DEAD Fixable Near-IR stain, ThermoFisher Scientific, cat #L34975, 5 min at room-temperature), fixation/permeabilization (eBioscience Foxp3/ Transcription Factor Staining Buffer Set, Cat #: 00-5523-00, 45 min at room-temperature) and, finally, intracellular staining of Helios (clone 22F6) and Foxp3 (clone 259D/C7) for 30 min at room-temperature. The LSRFortessa (BD Biosciences) was used for data collection and analysis was performed using FlowJo software (TreeStar). For TSDR analysis, 12 days after editing of Treg cells for OR1A1, cells were stained with anti-hCD4 and viability dye as described above followed by fixation with 0.5% paraformaldehyde (BioLegend, cat #420801) for 10 min at room-temperature. Fixed cells were washed twice with PBS containing 1% BSA, incubated in ice-cold methanol (100%) for 30 min on ice in order to permeabilize the cell membrane and thereafter stained intracellularly for Helios and Foxp3 as described above. After staining, cells were washed, resuspended in PBS with 1% BSA, and sorted as Foxp3⁻, Foxp3⁺Helios⁻, Foxp3⁺Helios^{lo} and Foxp3⁺Helios^{hi} (1×10⁶ cells/subset) on a BD FACSAria II cell sorter. Genomic DNA was isolated from each of the sorted populations as described above using the Qiagen Blood and Cell Culture DNA Mini Kit (Cat #: 13323) following the vendor recommended protocol. Bisulfite conversion and pyrosequencing of genomic DNA was performed by EpigenDx (assay ID ADS783-FS2) to quantify the methylation status of the FOXP3 gene region.

In vitro suppression of allogeneic or autologous T effector cells by Tregs: The suppressive function of either fresh or frozen Tregs was determined using a modified version of the Fastlmmune method developed by Canavan et al. (“A rapid diagnostic test for human regulatory T-cell function to enable regulatory T-cell therapy” Blood. 2012 Feb. 23; 119(8)) as well as a conventional in vitro suppression assay developed in-house. In case of frozen Tregs, cells were thawed 4 days prior to the start of the assays and rested overnight in Treg media containing IL-2 (300 units/mL). Overnight rested Tregs were re-stimulated in Treg media containing 10 μl/mL of ImmunoCult CD3/28/2 tetramer (StemCell Technologies, Cat #10970) and IL-2 (300 units/mL). One day prior to the start of the assays, frozen allogeneic or autologous PBMCs were thawed and labelled with cell trace violet (CTV) as follows: After washing PBMCs twice with PBS containing 0.1% BSA, cells were resuspended in the same buffer at 2.5×10⁷ cells/mL. An equal volume of CTV (12.5 μM) was slowly added to the cell suspension followed by an 8 min incubation at room-temperature in the dark. Labelled cells were washed three times with 10 volumes of Treg media and resuspended at 1×10⁶ cells/mL in the same media containing IL-2 (100 units/mL). On the following day, labelled PBMCs were washed once to remove residual IL-2 and resuspended at 5×10⁵ cells/mL in Treg media. Fifty microliters of this cell suspension were then added per well to two 96-well u-bottom plates (one for each assay). The pre-activated Treg cells were washed twice to remove IL-2 and tetramers and resuspended at 5×10⁵ cells/mL in Treg media. A 5-step 2-fold serial dilution of Treg cells was prepared in duplicates on a separate plate from which 50 μl was transferred to each of the two assay plates resulting in Treg:PBMC ratios of 1/1-1/16 with the last row containing PBMCs only. Dynabeads Human Treg Expander (ThermoFisher Scientific Cat #11129D, 7′500 beads/well) and APC-conjugated anti-human CD40L mAb (clone 24-31, BioLegend Cat #310805, diluted 1:50) were added to each well of the Fastlmmune Assay for a final volume of 0.2 mL per well. To the in vitro suppression assay plates, 0.1 mL of ImmunoCult CD3/28/2 tetramer (3 μl/mL) was added per well. After 7-12 hours of incubation at 37° C., the Fastlmmune assay was stopped and the cells were stained with mAbs to CD3 (clone HIT3a), CD4 (clone SK3), CD8 (clone SK1) and CD69 (clone FN50) (all mAbs were purchased from BioLegend). The level of Treg suppression was determined by flow cytometry comparing the expression (mean fluorescent intensity) of CD40L and CD69 on T effector cells cultured in the absence and presence of Tregs. The conventional suppression assay was terminated after 4 days of incubation and the cells were stained for CD3, CD4 and CD8 (see above for clones and vendor). CTV dilution was captured for CD4 and CD8 T effector cells by flow cytometry and the average number of cell divisions a cell in the original population had undergone was calculated using Division Index in FlowJo (TreeStar Inc).

### Example 2

### Effect of Continuous vs Discontinuous Stimulation on Treg Expansion

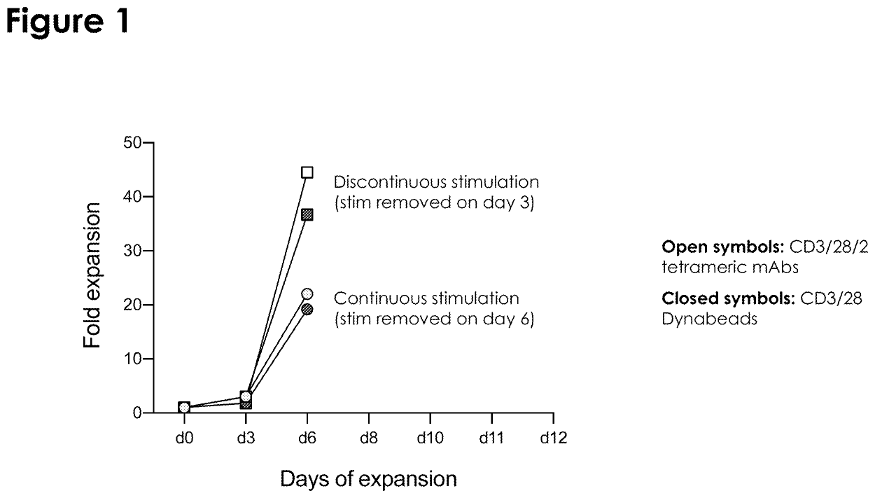

Assays were performed to test whether growth of Tregs can be accelerated by implementing a discontinuous stimulation regimen where periods of stimulation (i.e., when stimuli is present) are followed by periods of non-stimulatory conditions (i.e., when stimuli is absent). Human Tregs were FACS sorted as CD45RA^{+CD}25^{hi}CD127^{lo} from CD4+ T cells isolated from peripheral blood and then stimulated with either CD3/28/2 tetrameric antibody (open symbols) or CD3/28 coated magnetic Dynabeads in the presence of IL-2 and NAC. After 3 days of culture, the cells were washed, counted, and then divided into two separate wells containing media and IL-2 with or without tetrameric antibody (Ab)/Dynabeads. At day 6 of culture, cells were counted again, at which point discontinuously stimulated Tregs had grown ˜40-fold as compared to only ˜20-fold for continuously stimulated Tregs. (See FIG. 1.) The difference in growth between the two conditions was observed regardless of whether the cells were stimulated with tetrameric Ab or Dynabeads. (See id.)

### Example 3

### Effect of Continuous vs Discontinuous Stimulation on Treg Activation

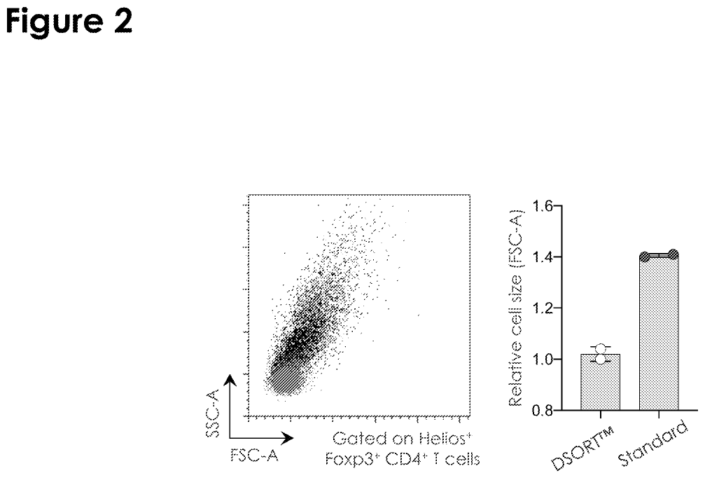

An assay was performed to assess what impact 3 days of non-stimulatory conditions have on the activation status of Tregs. Relative cell size, which is proportional to a cell's forward scatter (FSC) profile, is a commonly used indicator of the activation state of T cells cultured in vitro: naive cells have a smaller surface area and, hence, a lower FSC value relative to activated cells that have received T cell receptor (TCR) ligation. The results from this experiment show that Tregs that have been stimulated for 3 days followed by 3 days of non-stimulatory conditions have a significantly smaller surface area than Tregs that have been stimulated for 6 days straight. (See FIG. 2.)

### Example 4

### Effect of Extended Non-Stimulation on Treg Growth

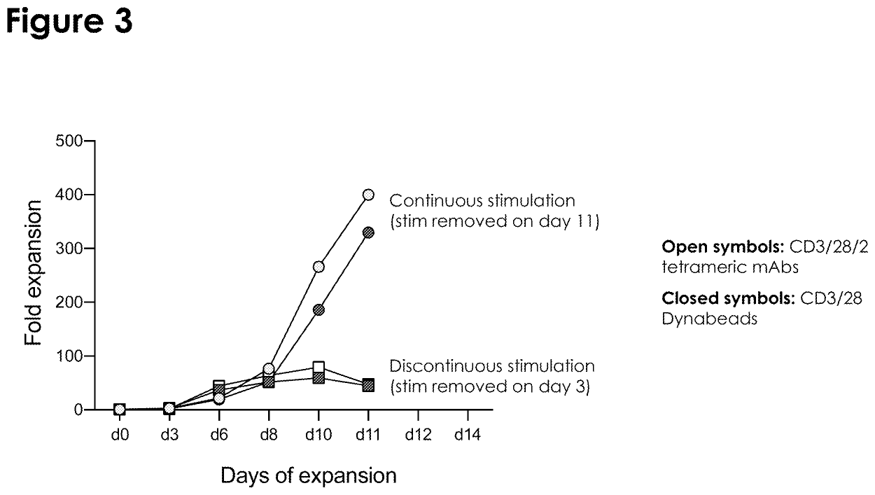

An assay was conducted to demonstrate how Treg growth is affected by extended periods of non-stimulatory conditions. As shown in FIG. 1, Treg growth is accelerated when stimulation is followed by a 3-day period of non-stimulatory condition. When the period of non-stimulatory condition is extended to 7 days, the growth, determined as fold-expansion between day 0 and 11, is reduced compared to standard conditions (continuous stimulation), even when Tregs are re-stimulated on day 10. (See FIG. 3.)

### Example 5

### Effect of Continuous vs Discontinuous Stimulation on Growth of Engineered Tregs

An assay was conducted to compare cell recovery after CRISPR engineering of Tregs subjected to discontinuous stimulation (DSORT™) or continuous stimulation (standard). Engineering was performed when cells had expanded ˜250-fold. DSORT™ Tregs had expanded ˜250-fold on day 8; standard Tregs had expanded ˜250-fold on day 11. Engineering was performed using single guide RNAs (sgRNAs) against a Treg-irrelevant gene, Orlal, that were transfected (electroporated) together with Cas9 protein into the cells.

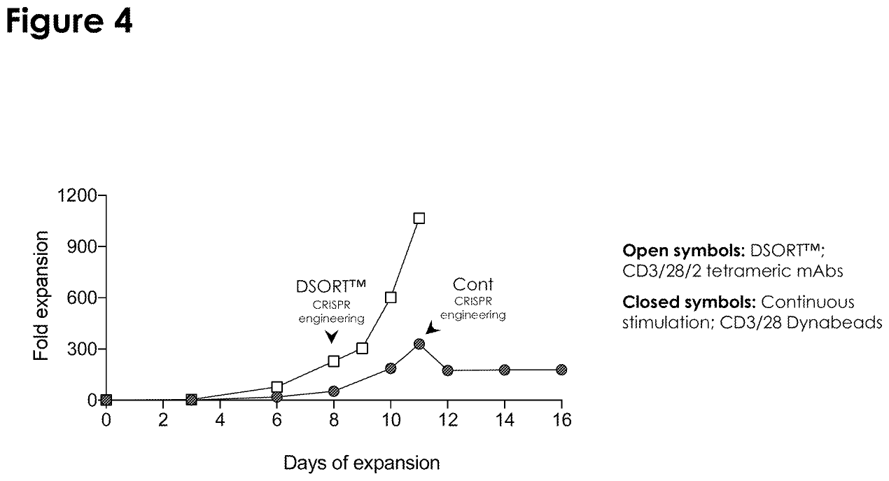

The number of viable Tregs was determined at the day of transfection, one day post-transfection, and then every 1-2 days for the remainder of the study. While DSORT™ Tregs (open squares) continued to grow another 3-fold following engineering, standard Tregs (closed circles) failed to recover to pre-engineering levels after transfection. (See FIG. 4.)

### Example 6

### Comparison of DSORT™ Expansion and Other Expansion Protocols

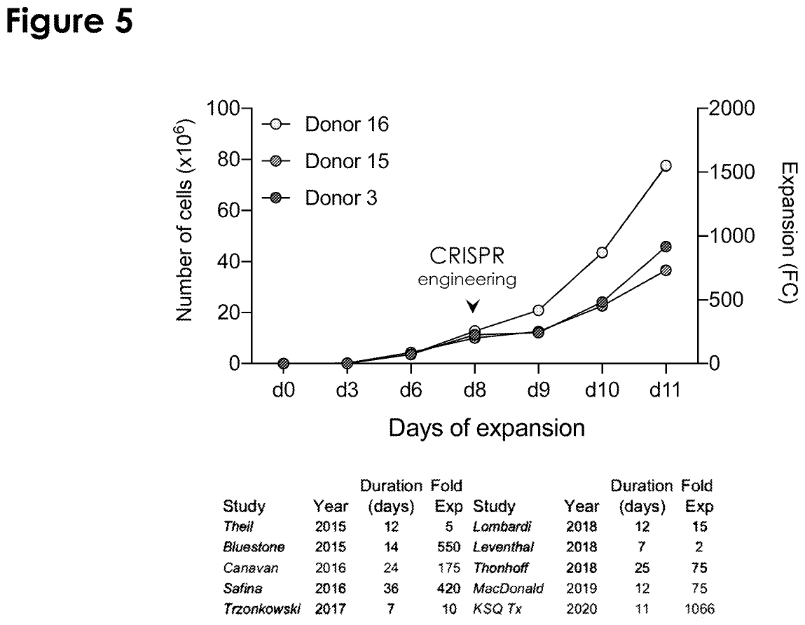

The growth rates of DSORT™ Tregs were compared to the growth rates of Tregs generated using publically available protocols for ex vivo expansion. The results are shown in FIG. 5. The top graph shows number of cells (left y-axis) and fold-expansion (right y-axis) over 11 days of ex vivo DSORT™ expansion of Tregs isolated from 3 different donors and engineered for a control gene (Or1a1) on day 8 of expansion. The bottom table shows corresponding fold expansions of Tregs from various studies published between 2015 and 2019. The Tregs in these published studies were cultured for longer than 11 days and were not engineered. The average fold-expansion of DSORT™ Tregs from the graph on top is included in the table (labeled as “KSQ Tx 2020) for reference. The results in this table demonstrate that the fold expansion of engineered DSORT™ Tregs was nearly double even the highest fold expansion observed in non-engineered Tregs subject to other expansion protocols.

### Example 7

### Effect of Continuous vs Discontinuous Stimulation on Stability of Tregs

Assays were performed to address how Tregs maintain lineage stability in vitro when subjected to discontinuous stimulation.

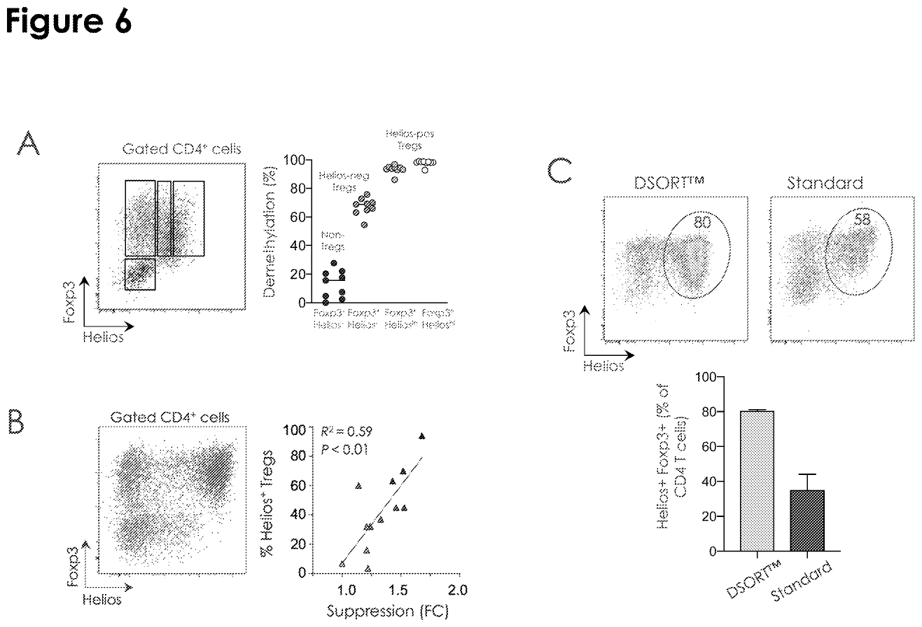

Helios is a transcription factor that reinforces the expression of Foxp3 in Tregs. Tregs that maintain stability from one cellular generation to the next have a fully demethylated Treg-specific demethylated region (TSDR, a locus within the Foxp3 gene), whereas the TSDR of Tregs that have converted from effector T cells in vitro (so-called induced Tregs) or are prone to destabilize under inflammatory conditions is partially methylated. The Tregs in this experiment were sorted into four subsets based on their expression of Foxp3 and Helios. Sorted cells were then analyzed using a DNA methylation assay (Pyrosequencing) to determine the level of methylation at the TSDR locus. The results, shown in FIG. 6A, demonstrate that only Helios+Tregs had fully demethylated TSDRs.

Tregs with various proportions of Helios+ cells were subjected to a conventional in vitro suppression assay that measures the ability of Tregs to suppress T effector cell proliferation. In FIG. 6B, the level of suppression (x-axis, depicted as fold-change, FC) was plotted against the proportion of Helios+ Tregs (y-axis). Tregs with a high proportion of Helios+ cells were superior to those with a low proportion of Helios+ cells in suppressing T effector cell proliferation. This data supports the importance of Helios as marker for Treg function.

The proportion of Helios+ Tregs generated using the DSORT™ protocol was compared to the proportion of Helios+ Tregs generated using standard (continuously stimulated) Tregs at day 12 of culture. The results, shown in FIG. 6C, demonstrate that a greater proportion of Tregs generated using the DSORT™ protocol were Helios+.

### Example 8

### Effect of Continuous vs Discontinuous Stimulation on Treg Suppressive Activity

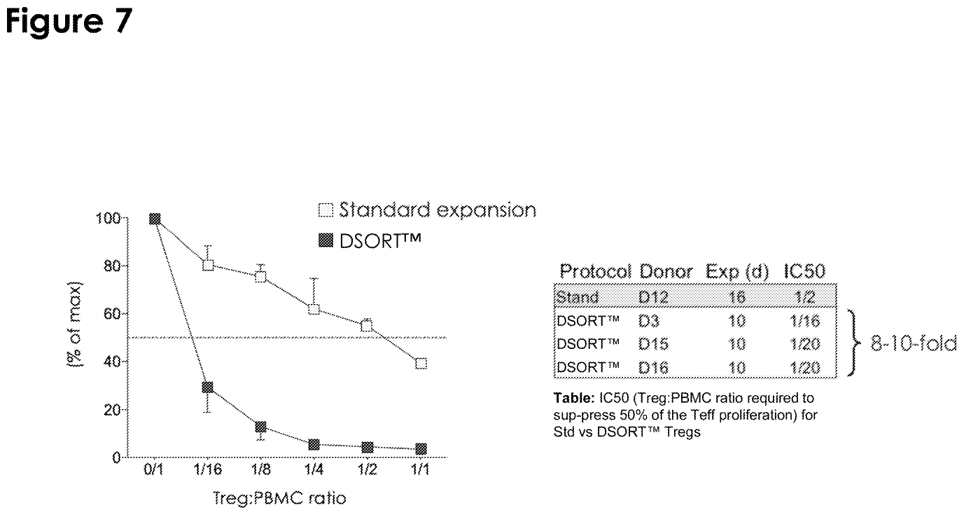

An assay was performed to compare the suppressive function of DSORT™ (filled squares) and standard (continuously stimulated) Tregs (open squares) (FIG. 7.) The graph presented in FIG. 7 shows the proliferation of CD4+ effector T cells (depicted as % of max proliferation on the y-axis) at various Treg to PBMC ratios (x-axis). The dashed horizontal line in the graph indicates the IC50 (Treg:PBMC ratio at 50% suppression). The table presented in FIG. 7 shows the IC50 values for DSORT™ and standard Tregs from multiple donors. Based on these data, the DSORT™ Tregs are at least 8 times as suppressive as standard Tregs.

All patents and publications cited herein are fully incorporated by reference herein in their entirety.

What is claimed is:

1. A method for expanding a population of regulatory T cells (Tregs), the method comprising:
(a) a first stimulating step comprising culturing a population of Tregs in the presence of a first stimulatory agent to produce a first stimulated population of Tregs; and
(b) a first resting step comprising continuing to culture the first stimulated population of Tregs in the absence of a stimulatory agent to produce a first rested population of Tregs.

2. The method of claim 1 further comprising:
(c) a second stimulating step comprising culturing the first rested population of Tregs in the presence of a second stimulatory agent to produce a second stimulated population of Tregs.

3. The method of claim 2 further comprising
(d) a second resting step comprising continuing to culture the second stimulated population of Tregs in the absence of a stimulatory agent to produce a second rested population of Tregs; optionally further comprising
(e) a third stimulating step comprising culturing the second rested population of Tregs in the presence of a third stimulatory agent to produce a third stimulated population of Tregs; optionally further comprising
(f) a third resting step comprising continuing to culture the third stimulated population of Tregs in the absence of a stimulatory agent to produce a third rested population of Tregs.

4. The method of claim 3 further comprising one or more additional stimulating step(s) to produce a further stimulated population of Tregs and/or one or more additional resting step(s) to produce a further rested population of Tregs.

5. The method of any one of claims 1-4, wherein the method of claim 1 further comprises genetically engineering the first rested population of Tregs, wherein the method of claim 2 further comprises genetically engineering the second stimulated population of Tregs; wherein the method of claim 3 further comprises genetically engineering the second rested population of Tregs, the third stimulated population of Tregs, or the third rested population of Tregs; or wherein the method of claim 4 further comprises genetically engineering the further stimulated population of Tregs or the further rested population of Tregs.

6. The method of any one of claims 1-5, wherein the method of claim 1 further comprises harvesting the first rested population of Tregs; wherein the method of claim 2 further comprises harvesting the second stimulated population of Tregs; wherein the method of claim 3 further comprises harvesting the second rested population of Tregs, the third stimulated population of Tregs, or the third rested population of Tregs; wherein the method of claim 4 further comprises harvesting the further stimulated population of Tregs or the further rested population of Tregs; or wherein the method of claim 5 further comprises harvesting the genetically engineered population of Tregs.

7. A method for expanding a population of regulatory T cells (Tregs), the method comprising:
(a) a stimulating step comprising culturing a population of Tregs in a media comprising a stimulatory agent to produce a first stimulated population of Tregs; and
(b) washing the stimulated population of Tregs to remove the media comprising the stimulatory agent to produce a washed population of Tregs; and
(c) culturing the washed population of Tregs in fresh media to produce a first rested population of Tregs.

8. The method of claim 7, wherein the fresh media does not comprise any stimulatory agent.

9. The method of claim 7 or 8, further comprising genetically engineering the first rested population of Tregs and/or culturing the first rested population of Tregs in a media comprising a stimulatory agent to produce a second stimulated population of Tregs.

10. A method for expanding a population of regulatory T cells (Tregs), the method comprising:
(a) a resting step comprising culturing a previously stimulated population of Tregs in the absence of a stimulatory agent to produce a first rested population of Tregs; and
(b) a stimulating step comprising adding a stimulatory agent to the first rested population of Tregs to produce a stimulated population of Tregs.

11. The method of any one of claims 7-10, further comprising genetically engineering the Tregs, optionally, genetically engineering the first rested population of Tregs.

12. The method of claim 5 or 11, wherein the method further comprises culturing the engineered population of Tregs.

13. The method of claim 12, wherein the culturing of the engineered population of Tregs occurs in the presence of a stimulatory agent, optionally wherein the method further comprises continuing to culture the engineered population of Tregs in the absence of the stimulatory agent.

14. The method of claim 12, wherein the culturing of the engineered population of Tregs occurs in the absence of the stimulatory agent, optionally wherein the method further comprises continuing to culture the engineered population of Tregs in the presence of a stimulatory agent.

15. A method for expanding a population of regulatory T cells (Tregs), the method comprising:
(a) genetically engineering a population of Tregs to produce a engineered population of Tregs;
(b) a stimulating step comprising culturing the engineered population of Tregs in the presence of a stimulatory agent to produce a stimulated engineered population of Tregs; and
(c) a resting step comprising continuing to culture the stimulated engineered population of Tregs in the absence of a stimulatory agent to produce a rested engineered population of Tregs.

16. A method for expanding a population of regulatory T cells (Tregs), the method comprising:
(a) genetically engineering a population of Tregs to produce an engineered population of Tregs;
(b) a resting step comprising culturing the engineered population of Tregs in the absence of a stimulatory agent to produce a rested engineered population of Tregs; and
(c) a stimulating step comprising culturing the rested engineered population of Tregs in the presence of a stimulatory agent to produce a stimulated engineered population of Tregs.

17. The method of claim 15 or 16, wherein prior to being genetically engineered, the population of Tregs is subject to a stimulating step comprising culturing the population in the presence of a stimulating agent; or wherein prior to being genetically engineered, the population of Tregs is subject to a stimulating step comprising culturing the population in the presence of a simulating agent and then a resting step comprising culturing the population in the absence of a stimulating agent; or wherein prior to being genetically engineered, the population of Tregs is subject to a stimulating step comprising culturing the population in the presence of a simulating agent, then a resting step comprising culturing the population in the absence of a stimulating agent, and then another stimulating step comprising culturing the population in the presence of a stimulating agent.

18. The method of any one of claims 11-17, wherein the genetic engineering occurs in the absence of a stimulatory agent.

19. The method of any one of claims 7-18, wherein the method further comprises harvesting the population of Tregs.

20. The method of any one of claims 1-19, further comprising obtaining the population of Tregs from a subject.

21. The method of any one of claims 1-19, further comprising obtaining the population of Tregs from thymus, peripheral blood, umbilical cord blood, or a tissue sample of a subject; or further comprising obtaining the population of Tregs from peripheral blood from a subject prior to the culturing of step (a).

22. The method of any one of claims 1-21, wherein the population of Tregs was obtained from thymus, peripheral blood, umbilical cord blood, or a tissue sample from a subject; or wherein the population of Tregs was obtained from peripheral blood from the subject.

23. The method of any one of claims 20-22, wherein the subject is human.

24. The method of any one of claims 1-23, wherein a tetrameric antibody complex is the stimulating agent in at least one of the stimulating steps and/or the culturing of the engineered population; optionally wherein the tetrameric antibody complex specifically binds to CD3, CD28, CD2, or a combination thereof.

25. The method of any one of claims 1-24, wherein an anti-CD3 antibody or antigen-binding fragment thereof and/or an anti-CD28 antibody or antigen-binding fragment thereof is the stimulating agent in at least one of the stimulating steps and/or the culturing of the engineered population.

26. The method of any one of claims 1-25, wherein CD3-binding and/or CD28-binding supermagnetic beads are the stimulating agent in at least one of the stimulating steps and/or the culturing of the engineered population.

27. The method of any one of claims 1-25, wherein the method does not use supermagnetic beads.

28. The method of any one of claims 1-27, wherein the same stimulatory agent is used throughout the method, or wherein at least two different stimulatory agents are used in the method; and/or wherein the stimulatory agent is present at the same concentration throughout all of the stimulating steps of the method, or wherein at least two different concentrations of stimulatory agent are used in the method.

29. The method of any one of claims 1-28, wherein the Tregs are cultured in the presence of IL-2, optionally wherein IL-2 concentration is reduced during the method, or optionally wherein IL-2 is present at a concentration of about 800 units/mL for about 7 days and then at about 300 units/mL.

30. The method of any one of claims 1-29, wherein the population of Tregs is cultured in the presence of a stimulatory agent for about 1 to about 5 days, then cultured in the absence of a stimulatory agent for about 1 to about 5 days, then cultured in the presence of a stimulatory agent for about 1 to about 5 days, and then genetically engineered; and/or wherein the population of Tregs is cultured in the presence of a stimulatory agent for about 3 to about 4 days, then cultured in the absence of a stimulatory agent for about 3 to about 4 days, then cultured in the presence of a stimulatory agent for about 1 to about 4 days, and then genetically engineered.

31. The method of any one of claims 1-30, wherein the population of Tregs is cultured in the presence and/or absence of a stimulatory agent according to Table A.

32. The method of any one of claims 1-31, wherein the Tregs are cultured in the presence of N-Acetyl-L-cysteine, optionally, wherein the N-Acetyl-L-cysteine is present at a concentration of about 5 mM in the culture.

33. The method of any one of claims 1-32, wherein the population of Tregs is genetically engineered when the population of Tregs has expanded about 250-fold.

34. The method of any one of claims 1-33, wherein the population of Tregs is genetically engineered about 6 to about 10 days after Tregs were obtained from a subject, optionally wherein the population of Tregs is genetically engineered about 7 days after Tregs were obtained from a subject.

35. The method of any one of claims 5, 6, and 11-34, wherein the genetic engineering comprises introducing a nucleic acid into the population of Tregs;
optionally, wherein the nucleic acid is a viral nucleic acid or wherein the nucleic acid is not a viral nucleic acid; and/or
optionally, wherein the nucleic acid encodes a protein, optionally wherein the protein is a heterologous protein, further optionally wherein the heterologous protein is a chimeric antigen receptor (CAR).

36. The method of any one of claims 5, 6, and 11-35, wherein the genetic engineering comprises introducing a gene-regulating system into the population of Tregs.

37. The method of claim 36, wherein the gene-regulating system comprises (i) a nucleic acid molecule; (ii) an enzymatic protein; or (iii) a nucleic acid molecule and an enzymatic protein; or
wherein the gene-regulating system comprises a nucleic acid molecule selected from an siRNA, an shRNA, a microRNA (miR), an antagomiR, or an antisense RNA; or
wherein the gene-regulating system comprises an enzymatic protein, and wherein the enzymatic protein has been engineered to specifically bind to a target sequence in one or more genes in the Tregs, optionally, wherein the enzymatic protein is a Transcription activator-like effector nuclease (TALEN), a zinc-finger nuclease, or a meganuclease; or
wherein the gene-regulating system comprises a nucleic acid molecule and an enzymatic protein, wherein the nucleic acid molecule is a guide RNA (gRNA) molecule and the enzymatic protein is a Cas protein or Cas ortholog, optionally wherein the Cas protein is a Cas9 protein.

38. The method of any one of claims 35-37, wherein the introducing uses electroporation or Ribonucleoprotein (RNP)-mediated methods.

39. The method of any one of claims 1-38, wherein the method does not use an artificial antigen presenting cell.

40. The method of any one of claims 1-39, wherein the method does not use rapamycin or wherein the method comprises using rapamycin.

41. The method of any one of claims 1-40, wherein the method increases the number of Tregs by at least 1000-fold in 11 days.

42. The method of any one of claims 1-41, wherein the method results in Tregs with a smaller surface area than Tregs that are cultured in the presence of a stimulating agent for 6 days; and/or wherein the method results in an increased proportion of Helios+Foxp3+ Tregs as compared to Tregs that are cultured in the presence of a stimulating agent for 6 days; and/or wherein the method results in Tregs with an increased ability to suppress proliferation of effector T cells (Teffs) as compared to Tregs that are cultured in the presence of a stimulating agent for 6 days; and/or wherein the method prevents overstimulation of the population of Tregs; and/or wherein the method reduces activation-induced cell death as compared to Tregs that are cultured in the presence of a stimulating agent for 6 days.

43. The method of claim 42, wherein the increased ability to suppress proliferation of Teffs is at least an 8-fold increased ability.

44. The method of any one of claims 1-43, wherein at least 75% of Helios expression in the Tregs is maintained.

45. The method of any one of claims 1-44, wherein the Tregs are Helios+.

46. The method of any one of claims 1-45, wherein the Tregs have a fully demethylated Treg-specific demethylated region (TSDR).

47. The method of any one of claims 1-46, further comprising cryopreserving the population of Tregs.

48. A population of Tregs produced by the method of any one of claims 1-47.

49. A cryoprsereved population of Tregs produced by the method of any one of claims 1-47.

50. The method of any one of claims 1-47, further comprising administering the population of Tregs to a subject.

51. A method of treating an autoimmune or inflammatory disease in a subject comprising administering to the subject an effective amount a population of Tregs obtained using the method of any one of claims 1-47 or the Tregs of claim 48.

52. A method of treating or preventing graft vs host disease (GVHD) in a subject comprising administering to the subject an effective amount a population of Tregs obtained using the method of any one of claims 1-47 or the Tregs of claim 48.

53. A method of decreasing an immune response in a subject comprising administering to the subject an effective amount a population of Tregs obtained using the method of any one of claims 1-47 or the Tregs of claim 48.

54. The method of any one of claims 50-53, wherein the population of Tregs is allogeneic to the subject; or wherein the population of Tregs is autologous to the subject.


SEQUENCE LISTING (7 sequences)

SEQ ID NO: 1 - protein, 450 aa, Artificial Sequence
  muromonab heavy chain
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, Artificial Sequence
  muromonab light chain
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 134 aa, Artificial Sequence
  recombinant human IL-2
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 4 - protein, 132 aa, Artificial Sequence
  aldesleukin
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 153 aa, Artificial Sequence
  recombinant human IL-7
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 6 - protein, 115 aa, Artificial Sequence
  recombinant human IL-15
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 7 - DNA, 20 bp, Artificial Sequence
  OR1A1 sgRNA
gctgaccagtaactcccagg